(19) United States
(12) Patent Application Publication
(10) Pub. No.: US 2005/0163760 A1
(43) Pub. Date: Jul. 28, 2005

(54) Use of cd34+ hematopoietic progenitor cells for the treatment of cns disorders

(76) Inventors: Nathalie Cartier-Lacave, Paris (FR); Patrick Aubourg, Boulogne-Billancourt (FR); Muriel Ashueur, Paris (FR); Sonia Benhamida, Paris (FR); Francoise Pflumio, Vitry Sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/501,738

(22) PCT Filed: Dec. 6, 2002
(86) PCT No.: PCT/IB02/05698

Related U.S. Application Data
(60) Provisional application No. 60/337,078, filed on Dec. 6, 2001.

(51) Int. Cl. A61K 48/00; C12N 5/08

(52) U.S. Cl. 424/93.21; 514/44; 435/372

(57) ABSTRACT

The present invention provides novel methods for delivering cells, particularly modified cells to the central nervous system (CNS). The purpose of this invention is to present a method that provides sustained delivery of a molecule to the central nervous system, thereby increasing the bioavailability of the molecule and lengthening the possible duration of treatment.

### FIELD OF THE INVENTION

The inventions relates to cell therapy, particularly the use of cell compositions enriched in hematopoietic progenitor cells to deliver therapeutic molecules to the central nervous system of a mammal, particularly of a human.

### BACKGROUND

Many diseases of the CNS in general lack effective treatment due to lack of adequate mechanism for the delivery of therapeutic molecules. Various drug delivery systems have been designed by using carriers such as proteins, peptides, polysaccharides, synthetic polymers, colloidal particles (i.e., liposomes, vesicles or micelles), microemulsions, microspheres and nanoparticles. These carriers, which contain entrapped pharmaceutically useful agents, are intended to achieve controlled cell-specific or tissue-specific drug release. Further efforts and research are being directed to develop and design novel systems of specific delivery to a target cell or tissue for the agents that cross biological barriers at relatively low rates.

In order to exert desired therapeutic or prophylactic effects, therapeutic molecules must reach brain cells and tissue. Intravenous administration will require their passage from the blood to the brain by crossing the microcapillary membranes of the cerebrovascular endothelium also called the blood-brain barrier. Briefly, the blood-brain barrier (BBB) is formed by a monolayer of tightly connected microvascular endothelial cells with anionic charges. This layer separates two fluid-containing compartments: the blood plasma (BP) and extracellular fluid (ECF) of the brain parenchyma, and is surrounded by astroglial cells of the brain. One of the main functions of the BBB is to regulate the transfer of components between the BP and the ECF. The BBB limits free passage of most agent molecules from the blood to the brain cells.

In general, large molecules of high polarity, such as peptides, proteins, (e.g. enzymes, growth factors and their conjugates, oligonucleotides, genetic vectors and others) do not cross the BBB. Therefore poor agent delivery to the CNS limits the applicability of such macromolecules for the treatment of neurodegenerative disorders and neurological diseases.

Several delivery approaches of therapeutic molecules to the brain circumvent the BBB. Such approaches utilize intrathecal injections, surgical implants and interstitial infusion. These strategies deliver an agent to the CNS by direct administration into the cerebrospinal fluid (CSF) or into the brain parenchyma (ECF).

Diffusion of macromolecules to various areas of the brain by convection-enhanced delivery is another method of administration circumventing the BBB. This method consists of: a) creating a pressure gradient during interstitial infusion into white matter to generate increased flow through the brain interstitium (convection-supplementing simple diffusion); b) maintaining the pressure gradient over a lengthy period of time (24 hours to 48 hours) to allow radial penetration of the migrating compounds (such as: neurotrophic factors, antibodies, growth factors, genetic vectors, enzymes, etc.) into the gray matter; and c) increasing drug concentrations by orders of magnitude over systemic levels.

In an attempt to provide a constitutive supply of drugs or other factors to the brain or other organs or tissues at a controlled rate, miniature osmotic pumps have been used. However, limited solubility and stability of certain drugs, as well as reservoir limitations, have restricted the usefulness of this technology. For example, controlled sustained release of dopa mine has been attempted by implanting dopamine encapsulated cells within bioresorbable microcapsules (McRae-Degueurce et al., 1988, Neurosci. Lett 92:303-309). However, controlled sustained release of a drug from a bioresorbable polymer may rely, e.g., on bulk or surface erosion, which may be due to various hydrolytic events. Erosion often relies on hydrolytic events which increase the likelihood of drug degradation, and complicates establishment of predictable release rates. Other disadvantages associated with pumps and resorbable polymers include finite loading capabilities and the lack of feedback regulation.

Another strategy to improve agent delivery to the CNS is by increasing the molecules' absorption (adsorption and transport) through the BBB and their uptake by the cells [Broadwell, Acta Neuropathol., 79:117-128, 1989; Pardridge et al., J. Phaimacol. Experim. Therapeutics, 255(2):893-899, 1990; Banks et al., Progress in Brain Research, 91:139-148, 1992; Pardridge, Fuel Homeostasis and the Nervous System, Edited by Vranic et al., Plenum Press, New York, 43-53, 1991]. The passage of agents through the BBB to the brain can be enhanced by improving either the permeability of the agent itself or by altering the characteristics of the BBB. Thus, the passage of the agent can be facilitated by increasing its lipid solubility through chemical modification, and/or by its coupling to a cationic carrier, or still by its covalent coupling to a peptide vector capable of transporting the agent through the BBB. Peptide transport vectors are also known as BBB permeabilizer compounds.

In other examples, direct administration to the CNS has been used to delivery molecules that would otherwise not pass the blood brain barrier. For example, polypeptides as well as viral vectors capable of directing the expression of a therapeutic polypeptide have been delivered intracerebrally and intrathecally. However, direct administration for the delivery of polypeptides has the evident convenience disadvantages due to repeated administration, and direct administration for the delivery of nucleic acids using viral vectors not been capable of achieving widespread transduction of cells beyond the site of administration.

Thus, the disadvantages of all of these approaches present a significant obstacle to the development of therapies of the treatment of CNS disorders, particularly those that involve a widespread population of neurons or glial cells.

Cell Transplantation

Cell transplantation, including ex vivo gene therapy has also been pursued as a therapeutic strategy, as for example in Bachoud-levi A C et al., (Lancet 2000, 356:1975-79) for Huntington's disease. However, the difficulties encountered vary greatly depending on the application and the cell source considerations, as exemplified in Table 1 (reproduced from Gage et al., Nature 392 (supp):18-24, 1998) below.

TABLE 1

| Cell source | Advantages | Disadvantages | Solution |
|---|---|---|---|
| Autologous | Immunologically | Limited supply; time | Cryopreserve; |
| | privileged; no ethical | constraints for donor and | multiply in vitro |
| | issues | host | |
| Allogeneic | Greater supply; few time | Cellular and humoral | Immunosuppress; |
| | constraints on donor | immunity; | encapsulate |
| | | ethical issues: fetal tissues | |
| Xenograft | Greater supply; no time | Cellular and humoral | Immunosuppress; |
| | constraints on donor | immunity; possible | encapsulate; |
| | | transfer of new virus | genetically mask |
| | | accross species | immunity |
| Cell line | Infinite supply; no time | Cellular and humoral | Immunosuppress; |
| (immortalized | contraints for donor or | tumorigenicity and | encapsulate; |
| or tumorigenic) | host; safety test and | neoplasia | genetically mask |
| | standardization simplified | | immunity |

Nevertheless, in view of the above-mentioned difficulties with other systems, alternative treatments for neurodegenerative diseases have emerged. As a general approach, cells have been transplanted into the area of neurodegeneration in an effort to reconstitute damaged neural circuits, and to replace lost neurons and neurotransmitter systems. Such treatments include transplantation of genetically engineered cells (see e.g., Breakefield, X. O. et al., 1989, Neurobiol. Aging 10:647-648; Gage, F. H. et al., 1987, Neuroscience 23:795-807; Horellou P. et al., 1990, Eur. J. Neurosci. 2:116-119; Rosenberg, M. B. et al., 1988, Science 242:1575-1578; Wolff, J. A. et al., 1989, Proc. Natl. Acad. Sci. USA 86:9011-9014) or fetal cells (see e.g., Bjorklund, A. et al., 1983, Acta. Physiol. Scand Suppl. 522:1-75; Dunnett, S. B. et al., 1990, in Brain Repair (eds. Bjorklund, A. et al.) Wenner-Gren Interiational Symposium Series 56:335-373 (McMillan Press, London); Isacson, O. et al., 1984, Nature 311;458-460; using porcine fibroblasts in U.S. Pat. No. 6,204,053.

In one strategy, engineered cells have been derived from cell lines or grown from recipient host fibroblasts or other cells and then modified to produce and secrete substances following transplantation into a specific site in the brain. For example, one group of researchers developed a biological system in which genetically engineered nerve growth factor-producing rat fibroblasts, when implanted into the rat striatum prior to infusion of neurotoxins were reported to protect neurons from excitotoxin-induced lesions (Schumacher, J. M. et al., 1991, Neuroscience 45(3):561-570). Another group which transplanted rat fibroblasts genetically modified to produce L-DOPA or dopamine into 6-hydroxydopamine lesions of the nigrostriatal pathway in rats reported that the transplanted fibroblasts reduced behavioral abnormalities in the lesioned rats (Wolff, J. A. et al., 1989, Proc. Natl. Acad. Sci. USA 86:9011-9014). As an alternative to genetically engineered cells, cells to be implanted into the brain can be selected because of their intrinsic-release of critical compounds, e.g., catecholamines by PC 12 cells and nerve growth factor by immortalized hippocampal neurons.

In other strategies, intracerebral neural grafting has emerged as a potential approach to CNS therapy. The replacement or addition of cells to the CNS which are able to produce and secrete therapeutically useful metabolites may offer the advantage of averting repeated drug administration while also avoiding the drug delivery complications posed by the blood-brain barrier (Rosenstein, Science 235:772-774, 1987). However, optimization of the survival of grafted cells has proved difficult, and no convenient and plentiful source of neurons is available.

Bone Marrow Transplantation

In yet other strategies, bone marrow transplantation (BMT) has been used to treat several genetic disorders that affect the CNS. The first group includes various lysosomal storage disorders with CNS involvement. In these disorders, deficiency of lysosomal enzyme affects primarily neurons (as in mucopolysaccharodisosis) or oligodendrocytes (as in metacromatic leukodystrophy or Krabbe disease). The rationale for BMT in the treatment of these disorders was that monocyte-derived cells from the donor can enter the brain, differentiate into microglia and/or perivascular macrophages and secrete normal lysosomal enzymes that can be recaptured by neurons or oligodendrocytes.

Similar reasoning was used initially by Moser H W et al., Neurology 34:1410-1417, 1984, to propose BMT in X-linked adrenoleukodystrophy (ALD). However, the protein to be delivered in X-linked adrenoleukodystrophy, the ALD protein, was a non-secreted protein, such that after the allogenic BMT, only microglia/perivascular macrophages expressed the normal ALD protein. The protein thus could not be secreted and recaptured by neurons and other glial cells. BMT for the treatment of ALD therefore likely represents a true form of “brain cell therapy”. Total BMT, referring to the transplantation of bone marrow cells without a purification or enrichment step, has also been demonstrated useful in the treatment of CNS disorders in multiple sclerosis (Burt et al, Inmunol. Today 1997, 18(12):559-561), autoimmune encephalomyelitis (van Gelder et al., Transplantation, 1996, 62(6):810-818, metachromatic leukodystrophy (Matzner et al. 2000, Gene Ther. 7(14):1250-1257), Fabry disease (Takenaka et al., 2000, PNAS USA 97(13):7515-7520), and gangliosidoses (Norflus et al., 1998, J. Clin. Invest. 101(9):1881-1888; and Oya et al., 2000, Acta Neuropathol. 99(2):161-168).

Transplanting total bone marrow presents several important disadvantages (Gage et al., 1998). Transplantation of whole bone marrow requires that several punctures in bone be made under anesthesia to obtain enough cells for transplantation. Furthermore, despite evidence suggesting that 1) monocytes can enter the brain and differentiate into perivascular macrophages and; 2) cells derived from the donor and having the morphological and histochemical characteristic of microglia can be recovered in the recipient mice after bone marrow transplantation, one does not know at which stage of differentiation (from early primitive HSC to already differentiated monocytic stage), hematopoietic cells enter the brain after bone marrow transplantation and differentiate into microglia.

Therefore, there is a need in the art for methods of delivering cells, nucleic acids and/or polypeptides to the CNS. There is accordingly also a need for methods of providing a convenient source of cells, particularly modified cells expressing a polypeptide of interest, capable of migrating to the CNS and providing or performing a desired biological function over a long period of time (e.g. months or years).

### SUMMARY OF THE INVENTION

The present invention provides novel methods for delivering cells, particularly modified cells, to the central nervous system (CNS). The purpose of this invention is to present a method that provides sustained delivery of a molecule to the central nervous system, thereby increasing the bioavailability of the molecule and lengthening the possible duration of treatment.

The invention involves providing a population of cells enriched in hematopoietic stem or progenitor or stem cells capable of migrating to the CNS upon administration to a subject at a site outside of the CNS. In preferred embodiments, the present invention provides a population of cells capable of differentiation into cells of the CNS, particularly microglia cells. Based on the characterization of populations of hematopoietic cells capable of giving rise to brain microglia expressing a desired polypeptide, the invention provides hematopoietic progenitor or stem cells and ex vivo therapies to provide cells that migrate to the CNS and differentiate into cell types found in the CNS. Moreover, in preferred embodiments, the populations of cells include hematopoietic progenitor or stem cells displaying the CD34 marker, allowing the cells to be conveniently separated using widely available equipment.

The invention is based on the inventors' demonstration that ex vivo genetic manipulation can be performed wherein human CD34+ cells and derived myelomonocytic cells obtained from ALD patients and transduced with a HIV-1 derived vector carrying the ALD cDNA can enter into the brain, differentiate into microglia and express a “therapeutic” protein. In a model of xenograft transplantation, it was demonstrated that myelomonocytic cells derived from human CD34+ cells can: 1) enter into the brain; 2) differentiate into microglia; 3) and express a “therapeutic” protein for several months, once these cells have been genetically modified ex vivo prior to transplantation.

In one aspect, the invention provides a method of administering a nucleic acid or protein of interest to the central nervous system of a mammal, comprising providing a composition enriched in hematopoietic progenitor cells or stem cells, and administering said composition to a mammal. Advantageously, at least of portion of said cells further comprise a nucleic acid of interest. The method provides particular advantages for the treatment of a mammal affected by or susceptible to being affected by a CNS disorder.

Disclosed is a method of delivering a nucleic acid sequence encoding a polypeptide of interest to a mammal, said method comprising: a) providing a composition enriched in hematopoietic progenitor cells or stem cells, preferably cells expressing the CD34 marker or cells capable of giving rise to cells expressing the CD34 marker, wherein at least a portion of said cells are recombinant cells comprising a nucleotide sequence encoding said polypeptide operably linked to expression control elements; and b) administering said composition to a mammal under conditions that result in the expression of the polypeptide of interest at a level that provides a therapeutic effect in said mammal. Furthermore, provided is a method of delivering a nucleic acid sequence encoding a polypeptide of interest to a mammal, said method comprising: a) obtaining cells from a human subject, said cells comprising hematopoietic progenitor cells or stem cells; b) isolating a hematopoietic progenitor or stem cell from said cells obtained from said subject; c) introducing a nucleic acid encoding a polypeptide of interest to said hematopoietic progenitor or stem cell; and d) administering said composition to a human subject affected by or susceptible to being affected by a CNS disorder under conditions that result in the expression of a polypeptide of interest at a level that provides a therapeutic effect in said mammal.

Encompassed also is a method for delivering a cell, preferably to the CNS of a mammal comprising: a) providing a composition enriched in hematopoietic progenitor cells or stem cells, said cells preferably expressing the CD34 marker or capable giving rise to cells expressing the CD34 marker; and b) administering said composition to a mammal. The administered cells will give rise to microglia cells in the CNS.

In preferred aspects of the methods of the invention, the at least of portion of said cells comprise a nucleic acid of interest. The nucleic acid of interest may encode a secreted or a nonsecreted protein. The cells of the invention are preferably transduced with a vector comprising a nucleic acid of interest operably linked to a promotor capable of effecting the, expression of said nucleic acid of interest in a hematopoietic cell. The vector is preferably a viral vector, most preferably a lentiviral vector.

In preferred aspects of the invention, at least a portion of the administered hematopoietic progenitor or stem cells are capable of migrating to the CNS and/or are capable of expressing the nucleic acid of interest in the CNS, and/or are capable of giving rise to cells of the CNS, preferably microglia.

Preferably human hematopoietic progenitor or hematopoietic stem cells are used in the present invention, most preferably human cells which are CD34+, or CD34+ and CD38−. It will be appreciated that it is also possible to use hematopoietic progenitor cells or stem cells capable of giving rise to cells which are CD34+, or more preferably CD34+ and CD38−. Preferably at least 10%, 20%, 50%, 75%, 90%, 95% or 99% of the cells, or essential all of the cells, in the cell composition administered to a mammal are hematopoietic progenitor or stem cells, and/or will express the CD34+ marker. The administered cells preferably comprise cells capable of reconstituting the immune system in a lethally irradiated host.

Most preferably, the cells are administrated to a subject by intravenous administration. Optionally, a subject is pre-treated in order to enhance engraftment of said progenitor or stem cells. Preferably, the cells administered to a subject are autologous cells.

As mentioned, typically the mammal to which the cells are administered according to the invention is affected by or susceptible to being affected by a CNS disorder. The methods of the invention will preferably result in a reduction in the severity of central nervous system damage or symptoms of a central nervous system disorder in a mammal. In most preferred aspects, CNS disorders include Alzheimer's disease, or any other CNS disorder characterized by diffuse neurodegeneration.

The methods and cells of the present invention can generally be used in a wide range of therapeutic applications. For example, cells may be used in order to replace or enhance a factor normally present in the CNS of a subject. The replacement may be of a function carried out by a subject's native microglia, as microglia are involved in many different biological functions, including examples as further discussed herein. However, cells of the invention are expected to be capable of expressing generally any suitable polypeptide such that replacement may also be of substantially any function or activity normally present in the CNS or carried out by a cell type present in the CNS. In other aspects, cells can be used to inhibit a function carried out by a subject's native microglia.

The invention further provides several advantageous therapeutic methods which can be carried out according to any of the methods of administering a nucleic acid or cell described herein. Disclosed in one aspect is a method of treating a central nervous system disorder in a manual comprising: a) providing a hematopoietic progenitor or stem cell; and b) administering said composition to a mammal affected by or susceptible to being affected by a CNS disorder, wherein said hematopoietic progenitor or stem cell gives rise to cells characterized by exhibiting decreased TNF-α secretion. In another aspect, the invention provides a method of treating HIV, optionally HIV dementia complex in a mammal comprising: (a) providing a hematopoietic progenitor or stem cell capable of expressing a polypeptide selected from the group consisting of: a mutated form of a CCR5 receptor, a mutated form of CXCR4, an CXCR4 ligand and a factor capable of inhibiting downstream signaling of CXCR4; and (b) administering said composition to a mammal affected by or susceptible to being affected by HIV, optionally HIV dementia complex.

Also provided is a method of treating a neurodegenerative disease in a mammal comprising (1) providing a hematopoietic progenitor or stem cell, preferably comprising a nucleic acid of interest; and (2) administering said composition to a mammal affected by or susceptible to being affected by a neurodegenerative disease, wherein said hematopoietic progenitor or stem cell migrates to the CNS and is capable of expressing a nucleic acid of interest in the CNS. Preferably the mammal to which the cells are administered is affected by or susceptible to being affected by CNS disease, such for example Alzheimer's disease. In one embodiment, said hematopoietic progenitor or stem cell gives rise to cells capable of modulating inflammation, e.g. interrupting inflammatory signaling cascades, in the CNS. In one embodiment, said hematopoietic progenitor or stem cell gives rise to microglia characterized by inhibiting or inactivating the complement pathway. Preferably said hematopoietic progenitor or stem cell comprises a nucleic acid of interest encoding a polypeptide acting as a C1 inhibitor. In another embodiment, said hematopoietic progenitor or stem cell comprises a nucleic acid of interest encoding a polypeptide acting as a COX-2 inhibitor. In other embodiments, said hematopoietic progenitor or stem cell gives rise to microglia capable of up regulating Aβ processing. In yet other embodiments, said hematopoietic progenitor or stem cell gives rise to microglia capable of inhibiting the binding of Aβ peptides to microglia type-A macrophage scavenger receptors. In another embodiment, said hematopoietic progenitor or stem comprises a nucleic acid of interest encoding a neuronal trophic factor.

The invention also encomposses a method of treating a central nervous system disorder in a mammal comprising providing a hematopoietic progenitor or stem cell; and administering said composition to a mammal affected by or susceptible to being affected by a CNS disorder, wherein said hematopoietic progenitor or stem cell gives rise to cells capable of activating NF-κB signaling. In another aspect, said hematopoietic progenitor or stem cell gives rise to cells capable of inhibiting NF-κB signaling.

All the methods of administering a nucleic acid of interest or a cell to the central nervous system of a mammal, particularly of a human, according to the present invention, methods which can be considered as method of treatment of an animal or a human body, could be converted as claims of use of a nucleic acid of interest or a cell in the preparation of a composition or a medicament for the treatment of a mammal, particularly of a human, affected by or susceptible to being affected by a CNS disorder wherein the characteristics of the claims methods can be included without limitations.

So, in another aspect of the present invention, the invention also comprises the use of a nucleic acid of interest for the manufacture of a composition for administration to a mammal, preferably a human, for the treatment of a subject affected by or susceptible to being affected by a CNS disorder, wherein said composition is a composition enriched in cells expressing the CD34 marker or cells capable of giving rise to cells expressing the CD34 marker, at least of portion of said cells comprising a nucleic acid of interest, and wherein at least a portion of said administered cells are capable of migrating to the CNS and expressing the nucleic acid of interest in the CNS of this subject.

In a preferred embodiment, the present invention comprises the use according to the present invention, wherein said administered cells are capable of giving rise to microglia in the CNS of said subject.

In still another aspect of the present invention, the present invention relates to the use of a nucleic acid sequence encoding a polypeptide of interest for the manufacture of a composition or a medicament for administration to a mammal, preferably a human, for the treatment of a subject affected by or susceptible to being affected by a CNS disorder under conditions that result in the expression of a polypeptide of interest at a level that provides a therapeutic effect in said subject, wherein said composition is a composition comprising hematopoietic progenitor or hematopoietic stem cells which have been isolated from cells comprising heematopoietic progenitor or stem cell obtained from a subject, and wherein a nucleic acid encoding a polypeptide of interest has been introduced to said isolated hematopoietic progenitor or stem cell.

In still another aspect of the present invention, the present invention relates to the use of cells for the manufacture of a composition or a medicament for administration to a mammal, preferably a human, for the treatment of a subject affected by or susceptible to being affected by a CNS, wherein said composition is a composition enriched in cells expressing the CD34 marker or cells capable of giving rise to cells expressing the CD 34 marker, and wherein at least a portion of said administered cells are capable of migrating to the CNS and giving rise to microglia

In a preferred embodiment, the present invention comprises the use according to the present invention, wherein said administration results in a reduction in the severity of central nervous system damage or symptoms of a central nervous system disorder.

In still another aspect of the present invention, the present invention relates to the use of a nucleic acid sequence encoding a polypeptide of interest for the manufacture of a composition or a medicament for administration to a mammal, preferably a human, for the treatment of a subject affected by or susceptible to being :affected by a CNS disorder under conditions that result in the expression of a polypeptide of interest at a level that provides a therapeutic effect in said subject, wherein said composition is a composition enriched in cells expressing the CD34 marker or cells capable of giving rise to cells expressing the CD34 marker, at least of portion of said cells being recombinant cells comprising a nucleotide sequence encoding said polypeptide operably linked to expression control elements.

In a preferred embodiment, the present invention comprises the use of a nucleic acid sequence encoding a polypeptide of interest according to the present invention, wherein at least a portion of said administered cells migrate to the CNS, give rise to microglia and express the nucleic acid of interest in the CNS of said subject.

In a more preferred embodiment, the present invention comprises the use of a nucleic acid sequence or cells according to the present invention, wherein said administered cells expressing the CD34 marker, cells capable of giving rise to cells expressing the CD34 marker, hematopoietic progenitor or hematopoietic stem cell differentiates into a microglia cell.

In another more preferred embodiment, the present invention comprises the use of a nucleic acid sequence according to the present invention, wherein at least a portion of said administered cells express the nucleic acid of interest in the CNS of said subject.

In another more preferred embodiment, the present invention comprises the use of a nucleic acid sequence or cells according to the present invention, wherein at least 20% of cells in said cell composition express the CD34+ marker, preferably at least 50%, 90% or essentially all of cells in said cell composition ekpress the CD34+ marker.

In another more preferred embodiment, the present invention comprises the use of a nucleic acid sequence or cells according to the present invention, wherein the administered cells are autologous to the subject to be treated.

In another more preferred embodiment, the present invention comprises the use of a nucleic acid sequence or cells according to the present invention, wherein administration is by intravenous administration.

In another more preferred embodiments the present invention comprises the use of a nucleic acid sequence or cells according to the present invention, wherein the subject to be treated is pretreated in order to enhance engraftment of said hematopoietic progenitor or hematopoietic stem cells.

In another more preferred embodiment, the present invention comprises the use of a nucleic acid sequence or cells according to the present invention, wherein said hematopoietic progenitor or hematopoietic stem cells or cells expressing the CD34+ marker are prior isolated.

In another more preferred embodiment, the present invention comprises the use of a nucleic acid sequence or cells according to the present invention, wherein said hematopoietic progenitor or hematopoietic stem cells are recombinant cells comprising a nucleic acid of interest.

In another more preferred embodiment, the present invention comprises the use of a nucleic acid sequence or cells according to the present invention, wherein said at least a portion of said hematopoietic progenitor or hematopoietic cells are transduced with a vector comprising a nucleic acid of interest operably linked to a promotor capable of effecting the expression of said nucleic acid of interest in said cell.

Preferably, said at least a portion of said hematopoietic progenitor cells or hematopoietic stem cells are transduced with a viral vector, particularly with a lentiviral vector.

In another more preferred embodiment, the present invention comprises the use of a nucleic acid sequence or cells according to the present invention, wherein said hematopoietic progenitor or hematopoietic stem cells express the CD34+ marker or are capable of differentiating into cells expressing the CD34+ marker.

In another more preferred embodiment, the present invention comprises the use of a nucleic acid sequence or cells according to the present invention, wherein said cells are hematopoietic progenitor cells or hematopoietic stem cells.

In another more preferred embodiment, the present invention comprises the use of a nucleic acid sequence or cells according to the present invention, wherein said administered cells comprises cells capable, in an animal model, of reconstituting the immune system in a lethally irradiated host.

In another more preferred embodiment, the present invention comprises the use of a nucleic acid sequence or cells according to the present invention, wherein said administered cells are human cells.

In another more preferred embodiment, the present invention comprises the use of a nucleic acid sequence or cells according to the present invention, wherein said nucleic acid encodes a nonsecreted or a secreted protein.

In another more preferred embodiment, the present invention comprises the use of a nucleic acid sequence or cells according to the present invention, wherein the CNS disorder which affects or which is susceptible to affect the subject is characterized by diffuse neurodegeneration, such for example Alzheimer's disease.

### DESCRIPTION OF THE FIGURES

FIGS. 1A to 1B: Phenotype and expression of ALD protein in bone marrow from NOD-SCID mouse transplanted with human ALD deficient CD34+ genetically engineered to express the ALD protein.

Cells were phenotyped by flow cytometry using monoclonal antibodies against human CD45 (FIG. 1A), CD19, CD15 (FIG. 1B), CD14 and CD11 (FIG. 1C) surface antigen markers.

FIG. 2: Bone marrow from NOD-SCID mouse transplanted with human ALD deficient CD34+ cells genetically engineered to express the ALD protein contains CD34+CD38− cells, indicating that early human hematopoietic progenitor cells were maintained in vivo.

FIGS. 3A to 3C: In situ hybridization of brain from NOD-SCID mouse transplanted with human ALD deficient CD34+ genetically engineered to express the ALD protein.

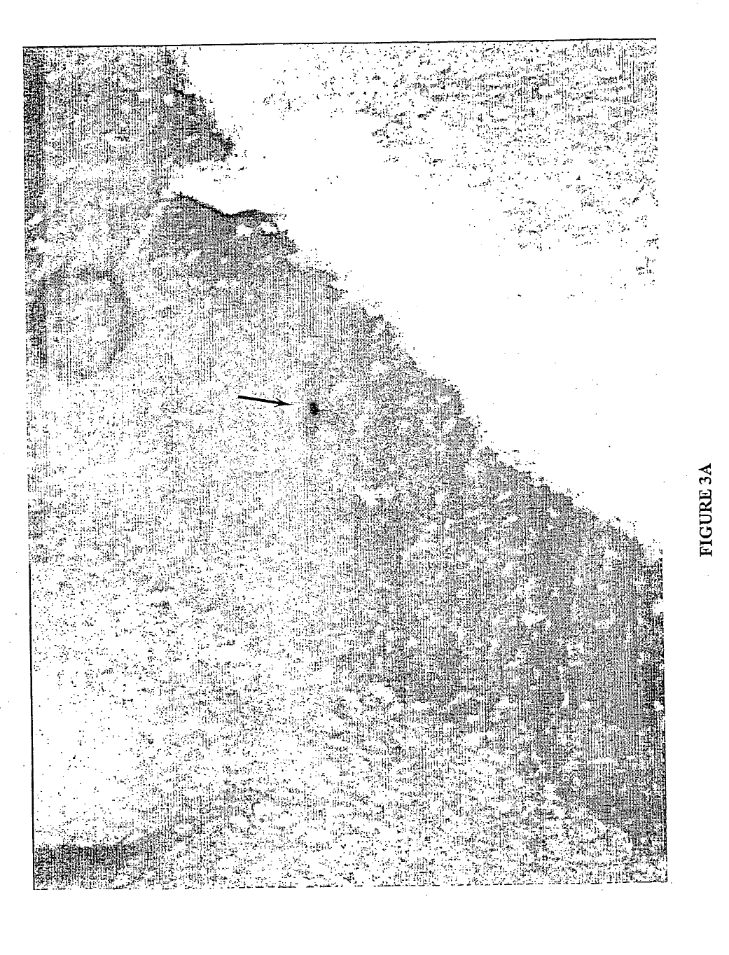

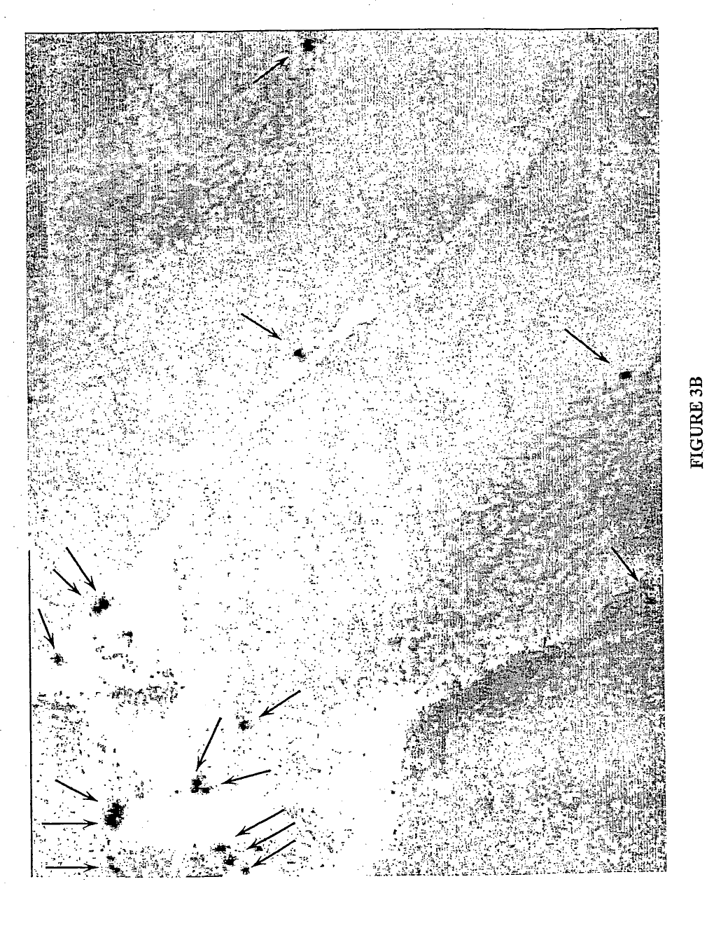

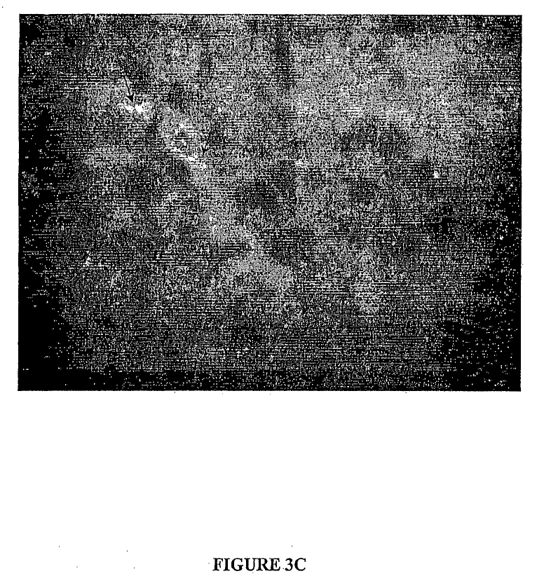

Cells containing human alu DNA sequences are present in the white matter of corpus callosum (FIG. 3A, arrow) and in the cerebellum (FIG. 3B, arrows). Cells strained with microglia marker (RCA, in green, fluorescein) express ALD protein (Cy3 in red) (FIG. 3c).

### DETAILED DESCRIPTION

Microglia

In the brain parenchyma, macrophages are called microglia. They were first recognized by Rio Hortega in 1932. Brain parenchyma macrophages/microglia are quite distinct from neurons, other glial cells (astrocytes, oligodendrocytes) and also distinct from macrophages associated with other part of the CNS (leptomeninges, choroid plexus, perivascular macrophages).

Microglia comprise a significant proportion of the nonneuronal cell population in the CNS: 5% in the white matter, up to 2% in the grey matter; up to 10-20% of all glial cells. Microglia are present in both grey and white matter. Some variation exists in the number of microglia cells among different brain regions but this does not reach one order of magnitude (Dobrenis K. Methods in Enzymology, 6:320-344, 1998; Perry V H and Gordon S. Trends Neurosci., 11:273-277, 1988; Perry V H. and Gordon S. Int. Rev. Cytol., 125:203-244, 1991; Perry V H. Macrophages in the central nervous system pp 87-101, R G Landes, Austin, 1994; Gonzales-Scarano F and Baltuch G. Annu. Rev. Neurosci., 22:29-240, 1999; Mittelbroon M. et al., Acta Neuropathol., 101:249-255, 2001). Perineuronal microglia are cells with somata that abut that of a neuronal perikaryon, often intimately wraps or covers a portion of the neuronal cell body.

Several markers recognize microglia including antibodies against surface antigens (HLA-DR; CD11a, CD11b and CD11c which are members of the β2 integrin family; leukocyte common antigen, Fcγ receptor, F4/80, MAC-1) and lectins (Griffonia simplicifolia I-B4). There is however no marker that specifically recognizes brain microglia and not macrophages located in other tissues. Adult microglia is often referred as “quiescent” or “resting” microglia, distinguishing it from “activated” microglia that arise in many pathological states. Resting microglia is ramified and downregulates the expression of most antigenic markers (ED1, CR3 complement receptor, MHC antigens) and functional indicators (cytokines) associated with macrophages in other tissues (Dobrenis, 1988, supra; Perry V H and Gordon S, 1988, supra; Perry V H and Gordon S, 1991, supra; Perry V H, 1994, supra; Gonzales-Scarano and Baltuch G, 1999, supra). In contrast to other organs where differentiated macrophages outnumber colonizing precursors, the majority of microglia (resting microglia) remains in an undifferentiated state towards immunologic response (Santambrogio L et al., Proc. Natl. Acad., Sci. USA., 98:6295-6300, 2001). When resting microglia becomes activated, the ramified appearance begins to withdraw, the cell body enlarges and cell reenters the cell cycle to undergo mitotic division.

Origin of Microglia

In contrast to other glial cells (astrocytes, oligodendrocytes) that are derived from neuroectoderm, observations have supported that microglia have a myelomonocytic origin. In particular, it has been observed that: 1) cells from bone marrow enter the CNS and adopt the morphology of microglia; 2) that monocytes invade the developing CNS and can transform to microglia (Hume D A et al., J. Cell Biol. 97:253-257, 1983;.Perry V H., Pontif. Acad. Sci. Scr. Varia, 59:281-295, 1985); 3) that microglia express antigens known to be partly or wholly restricted to cells of the monocytic lineage.

Evidence that bone-marrow derived cells enter the CNS was obtained from biochemical and histochemical analysis after bone marrow trans plantation (BMT) in mice and rats (Ting J P et al., Immunogenetics, 17:295-301,1983; Hickey W F and Kimura H. Science 239:290-292, 1988; Hickey W F et al., J. Neuropathol. Exp. Neurol., 51:246-256, 1992; Hoogerbrugge P M et al., Science 239:1035-1038, 1988; DeGroot, 1992; Lassmann H. and Mickey W F., Clin. Neuropathol., 12:284-285, 1993a; Lassmann H. et al., Glia 7:19-24, 1993b; Krall W J et al., Blood 9:2737-2748, 1994; Krivit W et al,. Cell Transplantation., 4:385-391, 1995; Eglitis M A and Mezey E. Proc. Natl. Acad. Sci. USA., 94:4080-4085, 1997). In these transplantation studies, donor bone marrow cells carried genes foreign for the donor, including ones for MHC, lysosomal enzyme, E. Coli galactosidase, SrY and λ phage.

Among these above referenced documents, the document Krall W J et al., (Blood 9:2737-2748, 1994) can be particularly cited. This publication discloses the study of macrophage and microglia replacement after murine autologous bone marrow transplantation with retrovirus-marked bone marrow. The authors indicate that, in the brain, 20% of the total microglia had been replaced with donor cells expressing the human giucocerebrosidase (GC) by 3 to 4 months after transplant.

Bone marrow transplantation (BMT) in rodents leads to a relatively rapid turnover of non parenchymal macrophages (20-40% turnover of perivascular macrophages 3 months after BMT). Turnover of resting and ramified microglia is slower (5 to 20% among different studies, 3 months after BMT). The turnover of macrophages is not restricted to perivascular macrophages as donor-derived ramified microglia has clearly been identified after bone marrow transplantation.

Most studies showing that bone marrow derived cells can differentiate into microglia were performed in rodents. For evident reasons, there are few data showing that the same process can occur when using human bone marrow cells. However it has been clearly demonstrated that male donor bone marrow derived cells can be recovered in the brain of female human brain after bone marrow transplantation (Unger E R et al., J. Neuropathol. Exp. Neurol., 52:460-470, 1993).

Myelomonocytic cells (and lymphocytes) are known to enter into the CNS via several routes: the lepto-meninges, choroid plexus and perivascular areas surrounding small vessels. The entry of these cells into CNS can be enhanced when the blood-brain-barrier is disrupted or modified, as it occurs when “inflammatory” changes take place into CNS (Dobrenis, 1988, supra; Perry V H and Gordon S; 1988, supra; Perry V H and Gordon S, 1991, supra; Perry V H, 1994, supra; Gonzales-Scarano and Baltuch, 1999, supra).

Nevertheless, despite evidence suggesting that 1) monocytes can enter the brain and differentiate in perivascular macrophages and; 2) cells derived from the donor and having the morphological and histo-immunochemical characteristics of microglia can be recovered in the recipient mice after bone marrow transplantation, until the present invention, one did not know at which stage of differentiation (from early primitive HSC to already differentiated monocytic stage), hematopoetic cells can enter into the brain after bone marrow transplantation and differentiate into microglia. Thus, previous experiments demonstrating in mice that brain microglia cells are derived from bone marrow cells have been performed using transplantation of total bone marrow cells. Using total bone marrow for treatment, however, presents significant disadvantages.

Functions of Microglia

Microglia interact with neurons, astrocytes and oligodendrocytes as well as extracellular elements in the CNS. Microglia have many functions (Perry V H and Gordon S., 1988, supra; Perry V H and Gordon S., 1991, supra; Perry V H, 1994, supra; Gonzales-Scarano and Baltuch, 1999, supra) several of which are further described as follows.

Among the known functions of microglia are important roles in phagocytosis, extracellular matrix catabolism and the production of growth factors during development as well as in the adult CNS. Thus microglia participate in the modeling of the CNS during the development and also act in a neuroprotective way against several types of injuries.

Microglia also have an important role in homeostasis. Microglia produce neurotransmitters and neuropeptides that interact with neurons and other glial cells.

Furthermore, microglia are involved in lipid turn-over, including ganglioside and phospholipid catabolism, a polipoprotein binding and secretion.

Microglia are also involved in inflammation, where activated microglia release cytokines (TNF-α, interferons, IL-1, IL-6), complement proteins, arachidonic acid (that potentiates NMDA receptor currents in neurons),: chemokines, cysteine, quinolinate, the amine Ntox which also potentiates NMDA receptor activation, neutral proteases, oxidative radicals, and nitric oxide that may contribute to death of neurons in several diseases. However, depending on the magnitude, timing and type of stimulus, activated microglia can also contribute to host defense and repair (Minghetti L. and Levi G. Prog. Neurobiol., 54:99-125, 1998; Gonzales-Scarano and Baltuch, 1999, supra; Akiyama et al., 2000). Thus, IL-6 plays a key role in regulating neuronal survival and function. IL-6 may cooperate with the high affinity neurotrophin receptor Trk. IL-6 can also act as an indirect immunosuppressant because it stimulates the pituitary-adrenal axis and elicits release of glucocorticoids. IL-6 also inhibits interferon-γ IL-1β and LPS (liposaccharide) induced synthesis of TNF-α (Akiyama et al., 2000). TNF-α may be. cytotoxic in brain trauma, multiple sclerosis and ischemic injury, but TNF-α can be trophic to rat hippocampal neurons, protects against glutamate, free radical and Aβ toxicity in cultured neurons. TNF-α, and is a potent stimulator of NF-κB, a transcription factor that increases the expression of survival factors such as calbindin, manganese-superoxide dismutase and the anti-apoptotic Bcl-2 protein (Akiyama et al., 2000).

Microglia are also involved in the immune response. Microglia are the principal immune cells in the CNS and play a role in antigen processing (APC-like cells). Microglia respond to traumatic injury or the presence of pathogens by migrating to the site of injury where they become activated and may proliferate. Like other macrophages, microglia release cytokines that rectute other cells (T and B cells) to the site of injury.

By providing cells that can give rise to microglia upon administration to a subject, any of these functions or properties of microglia can be provided, enhanced or modified to a subject in need thereof by delivering microglia to the CNS according to the invention. The function can be provided by administering unmodified cells (e.g. allogeneic) according to the methods of the invention thereby taking advantage of microglia's normal therapeutic capacities, or the function may be provided, enhanced or modified by administering cells which have been modified by the introduction of a therapeutic nucleic acid. As will be appreciated and further described herein, the introduction of a nucleic acid may be also useful to deliver a function not normally performed by microglia.

Dual Role of Microglia in the Pathogenesis of Neurodegenerative Diseases

According to the preferred methods of the present invention, microglia can be exploited in a therepeutic treatment in order to benefit from either or both of their dual roles in neurodegenerative disease. Preferred examples, further discussed below as well as in the section titled “Treatment”, include methods of treating neurodeneration such as in the exemplary cases Alzheimer's disease, Parkinson's disease, multiple sclerosis, and HIV dementia complex as well as in neuroprotection. Microglia may have deleterous or beneficial effects on the progression of several neurodegenerative diseases. Two examples are given as paradigms: CNS infection due to HIV and Alzheimer's disease.

HIV Infection

Individuals with HIV infection are predisposed to develop an opportunistic infections (toxoplasmosis) and neoplasmas (primary cerebral lymphoma) in the CNS. In addition, 20% of HIV-infected individuals develop a neurological syndrome, referred to as AIDS dementia complex or HIV dementia (HIVD), consisting of motor dysfunction, cognitive deterioration, and in later stages coma. This neurological syndrome is caused by HIV infection itself. In HIVD, there is scant evidence of infection of neurons. HIV enters the CNS via circulating lymphocytes or monocytes, which in turn transmit the virus to perivascular macrophages-microglia. Infected microglia survive for long periods of time and produce enough virus to maintain a cycle of new infections. Microglia express the β-chemokine receptor CCR5, which is the primary co-receptor for HIV (M-tropic isolates) with CD4. CCR5 is also the natural receptors for chemokines MIP-1α, MIP-1β and RANTES. Microglia express also CXCR4 (whose natural ligand is chemokine SDF-1) which can be used by HIV (SI isolates) to enter into these cells (Gonzales-Scarano and Baltuch, 1999, supra). In addition to their role in maintenance of infection of HIV within the brain, microglia are likely to have a direct role in neurotoxicity observed in HIV dementia. Among the candidate proteins secreted by HIV-infected microglia, the coat protein gp120 plays a role in activating indirectly NMDA receptors on neurons that leads to calcium influx and neuronal death (Bezzi P. et al., Nat. Neurosci., 4, 702-710, 2001). One role of microglia, and the TNF-α released by them, is to potentiate prostaglandin-dependent glutamate release from astrocytes that will activate NMDA receptors on neurons. Binding of gp120 on CXCR4 receptors at the surface of microglia evokes a large release of TNF-α which acts on the astrocyte signalling pathway to increase the production of prostaglandins (PgE2) and hence glutamate in the extracellular space.

Alzheimer Disease (AD)

Alzheimer disease (AD), the major cause (70%) of dementia in adult is a progressive neurodegenerative disorder that occurs in 5% of the population over 65 years of age. It is clinically characterized by a global decline in memory and other cognitive functions that leaves end-stage patients bedridden, incontinent and dependent on custodial care. Death occurs on average nine years after the diagnosis. The major risk for AD is increasing age and in the USA alone, there are currently over four millions patients with AD.

The major neuropathological changes in the brain of AD patients are neuronal death, particularly in regions related to memory and cognition and the presence of abnormal intra- and extra-cellular proteinaceous filaments. Intracellularly, bundles of paired helical filaments (PHF), composed largely of phosphorylated tau protein and referred to as neurofibrillary tangles, accumulate in large number in dying neurons. Extracellularly, insoluble aggregates of proteinaceous debris, termed amyloid, appear in the form of senile or neuritic plaques and cerebrovascular amyloid deposits. The amyloid deposits consist of aggregates of amyloid β-peptide (Aβ) isoforms. These are 39-42 residue peptides that are proteolytically derived from the large amyloid precursor (APP) by two proteases, β-secretase and α-secretase, and secreted by all cells. Cells secrete more Aβ40 than Aβ42 isoform that is less soluble and forms the major component of amyloid plaques. The fact that mutations in the APP gene are associated with familial AD is a strong indication of the importance of amyloid in the pathogenesis of the disease. The observation that activated microglia cluster around senile plaques suggests that microglia play an important role in the disease pathogenesis (Gonzales-Scarano and Baltuch, 1999, supra; Weninger S C and Yankner B A., Nat. Medecine, 7:527-528, 2001). Microglia in AD may have both deleterious and beneficial effects. Fibrillar Aβ peptides stimulate microglia leading to COX-2 activation, release of cytokines (TNFα, IL-1β, IL-6) and complement proteins that contribute to neurodegeration (Akkiyama et al., Neurobiology of aging. 21:383-421, 2000). High concentrations of Aβ40 or Aβ42 peptides do not damage neurons unless microglia are present. The HHQK domain within Aβ peptide provides a recognition site for microglial binding (Giulian D., Am. J. Hum. Genet., 65:13-18, 1999). However, microglia may have a beneficial effect in removal of the neurotoxic Aβ peptides. Microglia internalize Aβ fibrils by a type-A macrophage scavenger receptor (Paresce D M et al., Neuron 17:553-565, 1996), which is strongly expressed on activated microglia in the vicinity of senile plaques. The degradation of Aβ protein by microglia occurs via a secreted nonmatrix metalloprotease. The rate of Aβ degradation by microglia is however limited and the cells may be overwhelmed by the amount of Aβ present. In addition, it is not impossible that Aβ itself stimulates microglia to produce more Aβ by an autocrine loop.

Microglial Transplant as a Therapeutic Modality

The lineage of monocytes, microglia, and brain macrophages offer a simple and effective strategy for delivery of agents to the CNS in a global manner. As discussed herein, monocytes normally enter the CNS. This occurs during development but also in adulthood. Bone marrow transplantation experiments in rodents have demonstrated that turnover of parenchymal and resting microglia occurs, albeit at a slower rate than that for perivascular macrophages. Given the high degree of vascularization of the CNS, entry of microglia precursors can occur in a widespread manner and migrate into grey and white matter. The entry of these cells into CNS can be enhanced when the blood-brain-barrier is disrupted or modified, as it occurs when “inflammatory” changes take place into CNS.

Allogenic bone marrow transplantation has been used in humans to treat several genetic disorders that affect the CNS (Krivit W. et al., Cell Transplantation, 4:385-391, 1995; Krivit W. et al., Cur. Opin. Hematology, 6:377-382, 1999). The first group includes several lysosomal storage disorders with widespread CNS involvement. In these disorders, deficiency of a lysosomal enzyme affects primarily neurons (as in Hurler's disease) or oligodendrocytes (as in metachromatic leukodystrophy or Krabbe disease). The rationale for proposing BMT is these disorders is that monocyte-derived cells from the donor, can enter the brain, differentiate into microglia and/or perivascular macrophages and secrete a normal lysosomal enzyme that can be recaptured by neurons or oligodendrocytes lacking this lysosomal enzyme. BMT was shown to be efficacious in an other genetic CNS disorder, X-linked adrenoleukodystrophy (ALD) (Aubourg P. et al., N. Engl. J. Med., 323:1860-1866, 1990; Shapiro E. et al., Lancet 356:713-718, 2000). This disorder is characterized by progressive and widespread demyelination within the CNS, but in contrast to lysosomal storage disorders, the ALD gene encodes a non-secreted protein localized in the membrane of an intracellular organella (the peroxisome), which is a member of ATP-binding cassette transporter superfamily. Long-term efficacy of BMT has been confirmed in several CNS lysosmal storage diseases and ALD. Thus, replacement of endogenous microglia by normal donor-derived microglia can cure or halt CNS diseases characterized by widespread neuronal or glial pathology. BMT may allow the correction of CNS disease via two mechanisms: 1) the secretion of a “therapeutic” protein which is lacking or defective in neurons or other glial cells; 2) the replacement of endogenous defective nucroglia by microglia with normal function.

Efficacy of allogenic BMT is however markedly limited by the lack of HLA-identical donor and is associated with a significant mortality risk which is mainly due to rejection of the graft and/or severe graft-versus-host disease (GVH). Autotransplantation of hematopoietic stem cells (HSC) would circumvent these problems. In addition, since HSC can be genetically modified ex vivo prior to reinfusion. (Somia N. and Verma I M., Nature Rev., 1:91-99, 2000; Kay M A et al., Nat. Medecine. 7:33-40, 2001), any relevant “therapeutic” protein can be produced by HSC-derived microglia, in particular proteins that enhance CNS defense and repair. In addition, it is envisioned that HSC can be genetically manipulated in order to engineer microglia in which activation that may be deleterious in several CNS diseases is avoided.

In contrast to neural stem cells that are well characterized, primary HSC have not yet been fully characterized (in human as well as in mouse). However, various subpopulations of hematopoeitic cells from bone marrow containing HSC have been isolated, based on the presence/absence of antigen marker(s) on their surface. Several of the antigens and methods for obtaining enriched cell compositions or isolated cells are further described herein. This includes the sialomucin CD34 marker which allows the recovery of primitive HSC from bone marrow or from peripheral blood after stimulation with G-CSF (Krause D S et al., Blood 87:1-13, 1996). Allogenic transplantation or auto-transplantation of CD34+ cells are routinely performed in human patients and all relevant clinical and experimental protocols are designed, for CD34+ cells enriched by a variety of selection methods (Krause et al., 1996, supra). In rodents, long-term repopulation assays indicate that some stein cells that do not express detectable levels of CD34 antigen are also able to reconstitute bone marrow after transplantation in lethally irradiated recipient animals. This includes cells selected by high efflux of Hoechst 33342 dye (Goodell M A. et al., Nat. Medecine 3:1337-1345, 1997), by ALDH expression (Jones R J. et al., Blood 88:487-491, 1996), and CD34−/SRC cells (Bhatia M. et al., Nat. Medecine, 4:1038-1044). The evidence that CD34 negative cells also represent a population of HSC has however not been demonstrated in human.

As murine HSC and human CD34+ cells can be genetically modified ex vivo (for example after transduction with retrovirus or HIV-1 derived lentivirus vectors) (Case S C et al., Proc. Natl. Acad. Sci. USA, 96:2988-2993, 1999; Somia and Verma, 2000, supra; Kay et al., 2001, supra; Douglas J L et al., Hum. Gen. Ther., 12:401-413, 2001), the present invention provides the transplantation of human CD34+ cells that have been genetically modified ex vivo with the aim to express one or more specific transgenes in the microglia after transplantation. Additionally support for the feasibility is provided by Krall et al. (1994), supra, demonstrating that up to 20% of the total microglia of mouse brain can be replaced with donor cells expressing the human glucocerebrosidase enzyme (GC, a lysosomal enzyme which is deficient in the human disorder called Gaucher disease) after transplantation of syngenic bone marrow cells that were previously transduced ex vivo with a retroviral vector expressing the human GC. Additionally, data from Eglitis (1997), supra, demonstrated that microglia cells can express a transgene (the neomycine gene) after transplantation of bone marrow cells that were transduced ex vivo prior to transplantation.

Thus, according to the present invention, similar ex vivo genetic manipulation can be performed using compositions of cells that are enriched in or contain isolated populations of hematopoietic stem or progenitor cells for the delivery of a cell or polynucleotide to the brain. As described above and in the Examples, the present inventors have shown that human CD34+ cells and derived myelomonocytic cells can enter into the brain, differentiate into microglia and express a therapeutic protein. CD34+ cells from ALD patients were transduced with a HIV-1 derived vector carrying the ALD cDNA. ALD CD34+ cells were obtained from plasmapheresis after G-CSF was administered to patients. Up to about 50% of ALD deficient CD34+ cells were transduced by the HIV-derived lentiviral vector and expressed the ALD protein (all ALD CD34+ cells were ALD protein negative before the transduction owing to ALD gene mutation). The lentiviral-vector encoded ALD protein was shown biochemically to be functional in peroxisomes of transduced hematopoietic ALD cells by assessing the accumulation of VLCFAs, a deficiency caused by lack of functional ALD protein. These genetically modified human CD34+ cells as well as normal cord blood human CD34+ cells were transplanted (as a xenograft) into SCID-NOD mice. These mice have a severe combined immunodeficiency and transplantation of whole human bone marrow cells or human CD34+ cells was previously shown to reconstitute partially a hematopoietic cell system in these mice. Mice were engrafted with the transduced ALD deficient CD34+ cells in proportions ranging from 25% to 75% (% age of donor derived cells recovered in the bone marrow), and CD34/CD38− cells were found, indicating that early human hematopoietic progenitor cells were maintained in vivo. CD34+ cells from a tranplant recipient were also shown to contain CD68 positive cells expressing ADLP, indicating that long-term NOD/SCID repopulating cells derived from transduced ALD deficient CD 34+ cells were able to differentiate into monocytes/macrophages and express recombinent ALDP in bone marrow. Importantly, ALD positive cells were also recovered in the brain of the transplanted SCID-NOD mice. These cells expressed the donor-derived human Y chromosome, had the morphology of perivascular macrophages or ramified microglia and expressed RCA, a well recognized marker for microglia. ALD positive human microglia cells derived from normal cord blood human CD34+ cells or transduced ALD CD34+ cells were present in the grey and white matter of the SCID-NOD mice. ALDP was expressed in this way by human brain microglia for up to 4 months.

Definitions

By “vector” is meant any genetic element, such as a plasmid, phage, transposon, cosmid, chromosome, virus, virion, etc. The term includes cloning and expression vehicles, as well as viral vectors.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “coding sequence” or a sequence which “encodes” a particular protein, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genonic DNA sequences from prokaryotic or eukaryotic DNA, and even synthetic DNA sequences.

The terms DNA “control sequences” or “expression control element” refer to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter region” is used herein in its ordinary sense to refer to a nucleic acid region-comprising a DNA regulatory sequence, wherein the regulatory sequence is derived from a nucleic acid sequence which is capable of binding RNA polymerase and initiating transcription of a downstream (Y-direction) coding sequence.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

By “isolated” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition. For the purpose of describing the relative position of nucleotide sequences in a particular nucleic acid molecule throughout the instant application, such as when a particular nucleotide sequence is described as being situated “upstream”, “downstream” relative to another sequence, it is to be understood that it is the position of the sequences in the “sense” or “coding” strand of a DNA molecule that is being referred to as is conventional in the art.

By “isolated” when referring to a hematopoietic stem cell or progenitor cell, is meant that the indicated cell or cell type having a specified feature is present in the substantial absence of other cells not having said feature. Thus, an “isolated hematopoietic cell expressing a CD34 molecule” refers to a cell which is substantially free of other hematopoietic cells or different cell types that do not express the CD34 molecule; however, a composition of isolated cells may include some additional cells, so long as do not deleteriously affect the basic characteristics of the composition. Said isolated cell or cell composition may also include some cells of a different type as long as said cells express a specified feature, e.g. express a CD34 molecule.

The term “purified” does not require absolute purity; rather, it is intended as a relative definition. Purification of cells having a specified characteristic to at least one order of magnitude, preferably two or three orders, and more preferably 10, 100, 200 or 1000 orders of magnitude over that of a natural source of the cells is expressly contemplated. The term “purified” is further used herein to describe a cell or cell composition of the invention which has been separated from other cells not having a specified characteristic (e.g. hematopoietic type, a cell surface marker, progenitor cell, etc.). A cell composition can be said to be substantially pure when at least about 50%, preferably 60 to 75%, more preferably at least about 80, 90, 95 or 99% of the cells in a sample exhibits a specified characteristic.

The term “hematopoietic progenitor cell” , as used herein, refers to an undifferentiated cell derived from a hematopoietic stem cell, and is not itself a stem cell. Some progenitor cells can produce progeny that are capable of differentiating into more than one cell type. A distinguishing feature of a progenitor cell is that, unlike a stem cell, it has limited proliferative ability and thus does not exhibit self-maintenance. It is committed to a particular path of differentiation and will, under appropriate conditions, eventually differentiate into one of various cell types.

A “stem cell”, also referred to as a “pluripotent stem cell”, may be defined by its ability to give rise to progeny in all defined lineages. Stem cells are the multipotent self-renewing cells that sit at the top of the lineage heirarchy and proliferate to make differentiate cells types of a given tissue in vivo. Hematopoietic stem cells possessed the ability to fully reconstitute the immune system of a lethally irradiated host from which the cells are obtained. The hematopoietic stem cells give rise to all blood and immune cells. However, recent data suggest that stem cells from a given organ can also give progeny to cells that differentiate into cells from another organ, provided that the stem cells are in the appropriate microenvironment. Thus, bone marrow cells that contain hematopoietic stem cells can contribute to, astrocytes and neurons in the brain, skeletal muscle cells in tibialis anterior (Gussoni, E. et al., Nature, 1999, 401(6751):390-4; and Ferrari, G. et al., Science, 1998, 279(5356):1528-30), hepatic oval cells or hepatocytes in liver (Petersen, Science, 1999, 284(5417):1168-70; and Lagasse, E. et al., Nat. Med., 2000, 6(11):1229-34). Lin7⁻ c-kit^{POS} bone marrow cells can contribute to regeneration of myocytes in infarcted myocardium (Orlic, D. et al., Nature, 2001, 410(6829):701-5). Bone marrow derived circulating cells have the capacity to be a source of intimal smooth muscle like cells in murine allograft aortic transplant (Shimizu, K. et al., Nat. Med., 2001, 7(6):738-41).

The practice of the present invention will employ, unless otherwise indicated, conventional methods of, microbiology, molecular biology, recombinant DNA techniques and virology within the skill of the art. Such techniques are explained fully in the literature. See, e.g., Sambrook, et al., Molecular Cloning: A Laboratory Manual (Current Edition); Current Protocols in Molecular Biology (F. M. Ausubel, et al., eds., current edition); DNA Cloning: A Practical Approach, vol. I & 11 (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CR C Handbook of Parvoviruses, vol. I & 11 (P. Tijessen, ed.); Fundamental Virology, 2nd Edition, vol. I & 11 (B. N. Fields and D. M. Knipe, eds.).

As used in this specification and the appended claims, the singular forms “a”, “an” and “the” include plural references unless the context clearly dictates otherwise.

Obtaining Cell Populations

In contrast to neural stem cells that are well characterized, primary hematopoietic stem cells (HSC) have not yet been fully characterized (in human and mouse). As best, various subpopulations of hematopoeitic progenitor cells from bone marrow that contain HSC have been isolated, based on the presence/absence of antigen markers at their surface.

As used herein in the context of compositions enriched in hematopoietic progenitor and stem cells, “enriched” indicates a proportion of a desirable element (e.g. hematopoietic progenitor and stem cells) which is higher than that found in the natural source of the cells. In general, a natural source of cells will be processed so as to add or increase the proportion of the hematopoietic progenitor and stem cells. A composition of cells may be enriched over a natural source of the cells by at least one order of magnitude, preferably two or three orders, and more preferably 10, 100, 200 or 1000 orders of magnitude. Compositions enriched in hematopoietic progenitor or stem cells, or isolated hematopoietic progenitor or stem cells can be obtained for administration to a particular subject can be autologous cells or allogeneic cells. Hematopoietic progenitor or stem cells can also be derived from fetal or embryonic human tissue that is processed and/or cultured in vitro so as to increase the numbers or purity of primitave hematopoietic elements. In humans, CD34⁺ cells can be recovered from bone marrow or from blood after cytokine mobilization effected by injecting the donor with hematopoietic growth factors such as Granulocyte colony stimulating factor (G-CSF), granulocyte-monocyte colony stimulating factor (GM-CSF), stem cell factor (SCF) subcutaneously or intravenously in amounts sufficient to cause movement of hematopoietic stem cells from the bone marrow space into the peripheral circulation. Initially, bone marrow cells may be obtained from any suitable source of bone marrow, e.g. tibiae, femora, spine, fetal liver, and other bone cavities. For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include Hepes, phosphate buffers, lactate buffers, etc.

Cells can be selected using commercially available antibodies which bind to hematopoietic progenitor or stem cell surface antigens, e.g. CD34, using methods known to those of skill in the art. For example, the antibodies may be conjugated to magnetic beads and immunogenic procedures utilized to recover the desired cell type. The CD34 antigen, which is found on progenitor cells within the hematopoietic system of non-leukemic individuals, is expressed on a population of cells recognized by the nonoclonal antibody My-10 (i.e., express the CD34 antigen) and can be used to isolate stem cell for bone marrow transplantation. See Civin, U.S. Pat. No. 4,714,680, the disclosure of which is incorporated herein by reference. My-10 has been deposited with the American Type Culture Collection (Rockville, Md.) as HB-8483 and is commercially available from Becton Dickinson Immunocytometry Systems (“BDIS”) as anti-HPCA 1. However, using an anti-CD34 monoclonal antibody alone is not sufficient to distinguish between true pluripotent stem cells and other more differentiated cells, since B cells (CD19⁺) and myeloid cells (CD33⁺) make up 80-90% of the CD34⁺ population. Thus to improve progenitor or stem cell selection, a combination of monoclonal antibodies can advantageously be used to select human progenitor and stem cells. It is also possible to isolate CD34⁺ cells from monkeys.

Another antigen which may be used in selection is Class II HLA (particularly a conserved DR epitope recognized by a monoclonal antibody designated J1-43). HLA-DR is found on progenitor cells (although not on stem cells), and thus provides for some enrichment of progenitor activity by selecting for the marker, or for stem cells by negative selection. While these markers are also found in numerous lineage committed hematopoietic cells, they nevertheless allow at least a first improved enriched population of cells to be obtained.

The Thy-1 antigen can also be used for selection. Thy-1 is expressed on both progenitor cells and stem cells, and a particular subset of bone marrow cells meeting the criteria for stem cells has been found to express low levels of Thy (Thy^{lo}) (Baum et al., PNAS 89:2804-2808, 1992; Craig et al., 1993, J. Exp. Med 177: 1331-1342. A further selection antigen is c-kit, which is expressed on both hematopoietic stem and progenitor cells, although expression is gradually decreased upon maturation (Ogawa et al, 1991, J. Exp. Med 174: 63-71). These disclosures are incorporated herein by reference.

A sub-population of CD34⁺/CD38⁻ cells that contains more primitive HSC has been identified (Terstappen, 1991, Blood 77:1218-1227; Terstappen et al., 1994, Blood Cells, 20:45-63; Sutherland, 1989, the disclosures of which are incorporated herein by reference). Preferably, human hematopoietic stem cells are selected as being CD34+, CD38− in combination with lack of expression of the HLA-DR antigen (Verfaillie et al., 1990, J. Exp. Med., 172:509-520, the disclosure of which is incorporated herein by reference). While CD38 is expressed on 95-99% of bone marrow derived CD34+ cells, the CD38− fraction forms colonies with long term repopulating ability allowing a further purification if desired.

Additionally, a subpopulation of CD34⁻ cells that are positive for the dye Hoechst 33342 (Goodell M A et al., 1997, supra, the disclosure of which is incorporated herein by reference, was also shown to contain primitive HSC since their transplantation is able to reconstitute bone marrow in a host.

In mice, different markers are used: Lin−, Sca-1+, c-kit− and WGA for stem cells and Sca-1−, c-kit+ and WGA in progenitor cells. WGA, wheat germ agglutinin, is also expressed by both progenitor and stem cells, and again can be used to discriminate between progenitor and stem cells. Hematopoietic stem cells are WGAdim and hematopoietic progenitor cells are WGAbright (Ploemacher et al., 1993, Leukemia 7:120-130), Sca-1, stem cell antigen-1, is expressed on murine hematopoietic stem cells (Uchida and Weissman, 1992, J. Exp. Med 175:175-184) and to a lesser extent on progenitor cells (Spangrude et al., 1988, Science 241:58-62; and Spangrude et al., 1994, Ann. Rev. Med. 45:93-104). However, Sca- cells have been shown to have a short term repopulating ability when injected into sublethally irradiated mice suggesting that Sca- may be used to select committed hematopoietic progenitor cells. C-kit, mentioned above, can also be used for the selection of murine cells. Thy-1 also serves as a marker in mice and rats as well as in human. It has also been shown that rhodamine-123 can be used to distinguish stem cells from progenitor cells (stem cells appear dull when stained while progenitors are bright) (Baum et al., 1992; Fleming et al., 1993, J. Cell. Bio 122:897-902; Chaudhary and Robinson, 1991, Cell 66:85-94). The above disclosures are incorporated herein by reference.

In one example, a combination of anti-CD34 and anti-CD38 monoclonal. antibodies can be used to select those human progenitor stem cells that are CD34⁺ and CD38⁻. One method for the preparation of such a population of progenitor stem cells is to stain the cells with immunofluorescently labeled monoclonal antibodies. The cells then may be sorted by conventional flow cytometry with selection for those cells that are CD34⁺ and those cells that are CD38⁻. Upon sorting, a substantially pure population of stem cells results. (Becton Dickinson Company, published European Patent Application No. 455,482, the disclosure of which is incorporated herein by reference).

Additionally, negative selection of differentiated and “dedicated” cells from human bone marrow can be utilized, to select against substantially any desired cell marker. In known examples, this technique has yielded a population of human hematopoietic progenitor or stem cells with fewer than 5% lineage committed cells. See Tsukamoto et al., U.S. Pat. No. 5,061,620, the disclosure of which is incorporated herein by reference. For example, progenitor or stem cells, most preferably CD34+ cells, can be characterized as being any of CD3⁻, CD7⁻, CD8⁻, CD10⁻, CD14⁻, CD15⁻, CD19⁻, CD20⁻, CD33⁻, Class II HLA⁺ and Thy-1⁺.

Furthermore, a two-step purification of low density human bone marrow cells by negative immunomagnetic selection and positive dual-color fluorescence activated cell sorting (FACS) can be used. In one example a cell fraction was obtained that enriched 420-fold in pluripotent stem cells capable of initiating long-term bone marrow cultures (LTBMC) over unmanipulated bone marrow mononucleocytes (BMMNC) obtained after Ficoll-Hypaque separation, (Verfaillie et al., J. Exp. Med. 172, 509, 1990). Positive selection for small blast-like cells that are CD34 antigen positive but HLA-DR antigen negative was combined with a more extensive negative selection to deplete the population of CD2, CD19 and CD71-positive cells.

The isolation process can initially use a “relatively crude” separation to remove major cell families from the bone marrow or other hematopoietic cell source. If desired, large numbers of cells, namely major cell populations of the hematopoietic system such as T-cells, various lineages, such as B-cells, both pre-B and B-cells, granulocytes, myelomonocytic cells, and platelets, or minor cell populations, such as megakaryocytes, mast cells, eosinophils and basophils can be removed using initially magnetic bead separations. Optionally, in certain populations of progenitor cells, at least about 70%, usually 80% or more of the total hematopoietic cells can be removed using conventional methods. It is not essential to remove every dedicated cell class, particularly the minor population members, and the platelets and erythrocytes, at the initial stage.

The separation techniques employed should maximize the retention of viability of the fraction to be collected. For “relatively crude” separations, that is, separations where up to 10%, usually not more than about 5%, preferably not more than about 1%, of the total cells present having a selected marker, may remain with the cell population to be retained, various techniques of differing efficacy may be employed. The particular technique employed will depend upon efficiency of separation, cytotoxicity of the methodology, ease and speed of performance, and necessity for sophisticated equipment and/or technical skill. Procedures for separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate. Techniques providing accurate separation include fluorescence activated cell sorters, which can have varying degrees of sophistication, e.g. a plurality of color channels, low angle and obtuse light scattering detecting channels, impedance channels, etc.

As exemplary of the subject method, in a first stage after incubating the cells from the bone marrow for a short period of time at reduced temperatures, generally −10° to 10° C., with saturating levels of antibodies specific for T-cell determinants, the cells are washed with a fetal calf serum (FCS) cushion. The washed cells are then suspended in a buffer medium as described above and separated by means of the antibodies for the T-cell determinants.

Conveniently, the antibodies may be conjugated with markers, such as magnetic beads, which allow for direct separation, biotin, which can be removed with avidin bound to a support, fluorescers, e.g. fluorescein, which can use a fluorescence activated cell sorter, or the like, to allow for ease of separation of the T-cells from the other cells. Any technique may be employed which is not detrimental to the viability of the remaining cells.

Once the cells bound to the antibodies are removed, they may then be discarded. The remaining cells may then be incubated for a sufficient time at reduced temperature with a saturating level of antibodies specific for one or a mixture of cell differentiation antigens. The same or different mechanism for selecting for these cells as was used for removing the T-cells may be employed, where in the subject step, it is intended to use the unbound cells in subsequent stages.

The cells selected for as having the cell differentiation antigen are then treated successively or in a single stage with antibodies specific for the B-cell lineage, myelomonocytic, lineage, the granulocytic lineage, the megakaryocytic lineage, platelets, erythocytes, etc., although minor lineages may be retained, to be removed later. The cells binding to these antibodies are removed as described above, with residual cells desirably collected in a medium comprising fetal calf serum.

The residual cells are then treated with labeled antibodies selective but not specific for the stem cells, for mice the antibodies Sca-1 and Thy-1^{lo}, where the labels desirably provide for fluorescence activated cell separation (FACS). Multi-color analysis may be employed at this stage or previously. The cells are separated on the basis of an intermediate level of staining for the cell differentiation antigen, a high level of staining for Sca-1 and selected against dead cells and T-cells by providing for dyes associated with dead cells and T-cells as against stem cells. Other techniques for positive selection may be employed, which permit accurate separation, such as affinity columns, and the like. The method should permit the removal to a residual amount of less than about 1% of the non-stem or non-progenitor cell populations.

The particular order of separation is not critical to this invention, but the order indicated is preferred. Preferably, cells will be initially separated by markers indicating unwanted cells, negative selection, followed by separations for markers or marker levels indicating the cells belong to the stem cell population, positive selection.

Compositions having greater than 90%, usually greater than about 95%, of hematopoietic stem or progenitor cells may be achieved in this manner. Stem cells can be identified for example by having a low level of the Thy-1 cell differentiation antigen, being negative for the various lineage associated antigens and being positive for the Sca-1 antigen, which Sca-1 antigen is associated with clonogenic bone marrow precursors of thymocytes and progeny T-cells, or as already indicated, the human counterparts thereof.

However, the hematopoietic stem and progenitor cells that can be used according to the invention are not limited to cells expressing the aforementioned cell surface molecules. Any suitable assay for determining the capacity of cells as hematopoietic progenitor or stem cells can be used. In a first example, the stem cell activity of a human candidate cell is examined in vitro by testing its colony forming potential (McAdams, 1996, TIBTECH 12:341-349 and Ploemacher et al., Blood 79:834-837 (1992), the disclosures of which are incorporated herein by reference).

In preferred embodiments, hematopoietic stem cells according to the invention are characterized as having the ability to fully reconstitute the bone marrow of a lethally irradiated host. One assay for stem cell activity is an in vivo long-term marrow repopulating assay (MRA). For the assessment of mouse MRA, lethally irradiated mice can be transplanted with a bone marrow suspension and sacrificed 13 days after transplantation. The femoral cell content of the sacrificed mice is transplanted into secondary recipients and subsequently analyzed for colony forming units (CFU-S-12, CFU-GM capacity) (Ploemacher and Brons, Exp. Hematol. 16:21-26, 1988 and Hematol., 16:27-32, 1998, the disclosures of which are incorporated herein by reference). Cells generating colonies in secondary recipients are deemed to be derived from marrow seeded by stem cells in primary recipients. Another preferred assay is the long-term repopulating assay (LTRA) which identifies hematopoietic stem cell by allowing measurements of repopulating activity over longer times than the MRA wherein mice are sacrificed at 13 days (Jones et al, 1990, Nature 347:188-189; Li et al., 1992, J. Exp. Med 175:1443-1447; Morrison and Weissman, 1994, Immunity 1:661-673; Spangrude, 1995, Blood 85:1006-1016, the disclosures of which are incorporated herein by reference).

Animal models for the measurement of human MRA have also been developed, including a sheep in utero transplantation system in which human hematopoietic progenitor cells are transferred into the sheep fetus before the development of the ovine immune system. The presence of absence of human blood cells is then followed after the sheep is born. In another example, an assays test the ability of a candidate stem cell to repopulate the bone marrow of sublethally irradiated immune-deficient non-obese diabetic/SCID (NOD/SCID) mice. (See Lapidot T., et al., Science 255:1137, 1992; Vormoor et al., Blood 83:2489, 1994; Larochelle et al., Hum. Mol. Genet93. 4:163, 1995; Larochelle et al., Nat. Med. 2:1329, 1996, the disclosures of which are incorporated herein by reference).

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium comprising cell surface factors supporting the proliferation of stem cells. Stromal cells may be freed of hematopoietic cells employing appropriate monoclonal antibodies for removal of the undesired cells, for example, with antibody-toxin conjugates, antibody and complement, etc.

Modifying Cells

The hematopoietic stem cells may be genetically modified by introducing genetic material into the cells, for example using recombinant expression vectors.

A recombinant expression vector preferably comprises an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in eukaryotic expression systems preferably include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product.

Examples of suitable promoters which may be employed include, but are not limited to, TRAP promoter, adenoviral promoters, such as the adenoviral major late promoter; the cytomegalovirus (CMV) promoter; the respiratory syncytial virus (RSV) promoter; the Rous Sarcoma promoter; inducible promoters, such as the MMT promoter, the metallotlionein promoter; heat shock promoters; the albumin promoter; the ApoAI promoter; human globin promoters; viral thymidine kinase promoters, such as the Herpes Simplex thymidine kinase promoter; retroviral LTRs; ITRs; the β-actic promoter; and human growth hormone promoters. Preferably the promoter will be capable of driving expression of a gene operably linked thereto in a hematopoeitic cell; in one example the elongation factor 1α (EF 1α) promoter is used, which allows homogeneous expression in all hematopoietic cell types and particularly in NOD-SCID repopulating cells (Sirven, A. et al., Mol. Ther. 3, 438-448, 2001, the disclosure of which is incorporated herein by reference). The promoter also may be the native promoter that controls the gene encoding the polypeptide. These vectors also make it possible to regulate the production of the polypeptide by the engineered progenitor cells. The selection of a suitable promoter will be apparent to those skilled in the art.

The human hematopoietic stem cells thus may have stably integrated a recombinant transcriptional unit into chromosomal DNA or carry the recombinant transcriptional unit as a component of a resident plasmid. Cells may be engineered with a polynucleotide (DNA or RNA) encoding a polypeptide ex vivo, for example. Cells may be engineered by procedures known in the art by use of a retroviral particle containing RNA encoding a polypeptide.

Various methods are available for genetically modifying donor cells prior to implantation into a recipient subject. Suhr, S. T. and Gage, F. H., 1993, Arch. Neurol. 50(11):1252-1268; Gage, F. H. et al., 1987, Neuroscience 23(3):795-807. These methods include direct DNA uptake (transfection), and infection with viral vectors such as lentivirus, retrovirus, herpes virus, adenovirus, and adeno-associated virus vectors. Suhr, S. T. et al., 1993, Arch. Neurol. 50:1252-1268. Transfection can be effected by endocytosis of precipitated DNA, fusion of liposomes containing DNA or electroporation. Suhr, S. T. et al., 1993, Arch. Neurol. 50:1252-1268. Another method of transfecting donor cells is through the use of a “gene gun”. In this method, microscopic DNA-coated particles are accelerated at high speeds through a focusing tube and “shot” or injected into cells in vitro (Klein, R. M. et al., 1992, Biotechnology 24:384-386; Zelenin, A. V. et al., 1989, FEBS Lett., 244:65-67) or in vivo (Zelenin, A. V. et al., 1991, FEBS Lett., 280:94-96). The cells close around the wound site and express genes carried into the cell on the particles. All of the above-referenced are incorporated herein by reference.

Retroviral vectors typically offer the most efficient and best characterized means of introducing and expressing foreign genes in cells, particularly mammalian cells, These vectors have very broad host and cell type ranges, integrate by reasonably well understood mechanisms into random sites in the host genome, express genes stably and efficiently, and under most conditions do not kill or obviously damage their host cells. The methods of preparation of retroviral vectors have been reviewed extensively in the literature (Suhr, S. T. and Gage, F. H., 1993, Arch. Neurol. 50(11):1252-1258; Ray, J. and Gage, F. H., 1992, Biotechniques 13(4):598-603; Anderson, W. F., 1984, Science 226:401-409; Constantini, F. et al., 1986 Science 233:1192-1194; Gilboa, E. et al., 1986, Biotechniques 4:504-512; Mann, R. et al., 1983, Cell 33:153-159; Miller, A. D. et al., 1985, Mol. Cell Biol. 5:431-437; and Readhead, C. et al., 1987, Cell 48:703-712) and are now in common use in many laboratories. Suitable vectors and improved methods for production of recombinant retroviral vectors are also provided in U.S. Pat. No 6,013,516. Other techniques for producing genetically modified cells are described in detail in PCT publication WO 95/27042. All of the above-referenced are incorporated herein by reference.

Retroviruses from which the retroviral plasmid vectors hereinabove mentioned may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus (e.g. HIV-1), adenovirus, Myeloproliferative Sarcoma Virus; and mammary tumor virus. In one embodiment, the retroviral plasmid vector is MGIN, derived from murine embryonic stem cells.

In preferred embodiments, lentiviral vectors are used due to their ability to introduce genes into non-dividing or post-mitotic cells. Lentiviral vectors also do not suffer from low viral titer limitations as do certain other vectors. Lentiviruses are complex retroviruses, which, in addition to the common retroviral genes gag, pol and env, contain other genes with regulator or structural function. The higher complexity enables the virus to regulate its life cycle, as in the course of latent infection. A typical lentivirus is Human Immunodeficiency Virus (HIV), the etiologic agent of AIDS. In vivo, HIV can infect macrophages which are terminally differentiated cells that rarely divide. In vitro, HIV can infect primary cultures of monocyte-derived macrophages (MDM), and HeLA-Cd4 or T-lymphoid cells arrested in the cell cycle by treatment with aphidicolin or gamma irradiation. Infection of these cells is dependent on the active nuclear import of HIV preintegration complexes through the nuclear pores of the target cells. This occurs by the interaction of multiple, partly redundant, molecular determinants in the complex with the nuclear import machinery of the target cell. Identified determinants include a functional nuclear localization signal (NLS) in the gag MA protein, the karyophilic virion-associated protein vpr, and a C-terminal phosphotyrosine residue in the subset of the gag MA protein.

The recently described HIV based lentiviral vector has been shown to be efficient in integrating into non-cycling cells (Verma, Nature 389:239-242, 1997). Studies to determine the usefulness of this vector have been performed by Choi and Gewirtz (1998, Blood 92:468a). To obtain better expression, Uchida et al. (1998, PNAS USA 95:11939-11944) successfully utilized a HIV-based vector system that also expressed the viral transcription co-factor tat that is critical for high expression of the HIV LTR. A hybrid HIV/murine stem cell virus (NSCV) vector has also been developed where in the original internal CMS enhancer/promoter is removed and the U3 region of the HIV LTR is partially replaced by the U3 region of the MSCV LTR for increased safety with a high transduction efficiency (U.S. Pat. No. 6,218,186). All of the above-referenced are incorporated herein by reference.

A lentivirus vector may be an attenuated virus that has been modified so that it is incapable of causing disease of pathology in a host animal or cell (i.e. it encompasses virus that are incapable of causing or cause reduced cytopathic effects in viral cultures). Viral particles may be capable of some degree of infection and gene expression, but are not able to produce disease or productive infection.

Vectors for gene transfer into hematopoietic cells are also reviewed in Elwood, Leuk. Lymphoma 2001, 41 (5-6):465-482, incorporated herein by reference.

It is also possible to use vehicles other than retroviruses to genetically engineer or modify the hematopoietic stem cells. Genetic information of interest can be introduced by means of any virus which can express the new genetic material in such cells. For example, SV40, herpes virus, adenovirus, adeno-associated virus and human papillomavirus can be used for this purpose. Other methods can also be used for introducing cloned eukaryotic DNAs into cultured manmmalian cells, for example, the genetic material to be transferred to stem cells may be in the form of viral nucleic acids.

In addition, the expression vectors may contain one or more selectable marker genes to provide a phenotypic trait for selection of transformed cells such as dihydrofolate reductase or neomycin resistance.

The hematopoietic cells may be transfected through other means known in the art. Such means include, but are not limited to transfection mediated by calcium phosphate or DEAE-dextran; transfection mediated by the polycation Polybrene; protoplast fusion; electroporation; liposomes, either through encapsulation of DNA or RNA within liposomes, followed by fusion of the liposomes with the cell membrane or, DNA coated with a synthetic cationic lipid can be introduced into cells by fusion.

The present invention further makes it possible to genetically engineer human hematopoietic stem or progenitor cells in such a manner that they produce polypeptides, hormones and proteins not normally produced in human hematopoietic cells or in microglia or other cells of the CNS in biologically significant amounts or produced in small amounts but in situations in which regulatory expression would lead to a therapeutic benefit. For example, the hematopoietic stem cells could be engineered with a gene that expresses a molecule that specifically inhibits neurodegeneration. Alternatively the cells could be modified such that a protein normally expressed will be expressed at much lower levels. These products would then be secreted into the surrounding media or purified from the cells. The human hematopoietic stem cells formed in this way can serve as continuous short term or long term production systems of the expressed substance. These genes can express, for example, hormones, growth factors, matrix proteins, cell membrane proteins, cytokines, adhesion molecules, “rebuilding” proteins important in tissue repair. The expression of the exogenous genetic material in vivo is often referred to as “gene therapy”.

Nucleic Acids

As will be appreciated by the skilled person, according to the invention, the cells may be engineered to express any suitable nucleic acid sequence. In one aspect, a nucleic acid sequence may serve to express a nucleic acid acting directly on a biological target, such as in an antisense or ribozyme treatment. In other aspects, said nucleic acid sequence may encode a polypeptide. As used herein, the terms peptide and polypeptides are used interchangeably, as polypeptides of essentially any length may be used in accordance with the present invention. Polypeptides may be full-length polypeptides or fragments thereof suitable for a particular application (e.g. capable of restoring a biological activity, inhibiting a biological activity). Polypeptides may be secreted or non-secreted polypeptides.

A nucleic acid can encode a functionally active polypeptide or an inhibitor, e.g. of a target polypeptide or an inhibitor of a binding event. For example, a polypeptide may be a dominant negative mutant polypeptide. Non-limiting examples of nucleic acids that can be expressed include nucleic acids encoding neuropeptides, neurotransmitters, enzymes involved in biosynthesis, proteins involved in intracellular signalling pathways, antibodies, pro- or anti-inflammatory molecules (for example cytokines), and receptors. For example, viral vectors have been developed encoding enzymes responsible for doparine biosynthesis (Freese et al., 1997, Epilepsia 38 (7):759-766) and the GluR6 excitatory amino acid receptor subtype (Bergold et al., 1993, PNAS USA 90: 6165-6169). In certain applications, nucleic acids may allow detection of virions and/or detection of transgene expression. Nucleic acids may encode detectable marker polypeptides, such as a fluorescent protein (ex. GFP) or another detectable polypeptide such as β-galactosidase. Other non-limiting examples of genes suitable for use according to the invention include anti-apoptotic genes such as bcl-2, interleukin-1 converting enzyme, crmA, bcl-xl, FLIP, survivin, IAP, ILP; genes which provides target cells, preferably tumor cells, with enhanced susceptibility to a selected cytotoxic agent, such as the herpes simplex virus thymidine kinase (HSV-tk), cytochrome P450, human deoxycytidine kinase, and bacterial cytosine deaminase genes (see also Springer and Niculescu-Duvaz, 2000, J. Clin. Invest., 105:1161-1167). Also included are polypeptides which reduce glutamate toxicity, and polypeptides with act as calcium buffers or binding protein such as calbindin. Also encompassed are polypeptides capable of inhibiting the activity of an enzyme. For example, encompassed in Alzheimer's disease are a polypeptide capable of inhibiting or reducing the formation of Aβ production, a polypeptide capable of modifying APP processing, a polypeptide capable of stimulating or generally increasing α-secretase cleavage activity, a polypeptide capable of inhibiting the β-secretase pathway, a polypeptide capable of inhibiting the γ-secretase pathway, or a polypeptide capable of inhibiting tau protein hyperphosphorylation.

Other examples of nucleic acids that can be used with the present invention include nucleic acids coding for growth factors or neurotrophic factors, including but not limited to genes encoding: acidic fibroblast growth factor (aFGF; FGF-1); glial cell line-derived neurotrophic factor; brain-derived neurotrophic factor; nerve growth factor; TGF-α, extracellular matrix proteins (collagens, fibronectins, integrins); ornithine amino transferase; prostaglandin synthesis regulation proteins; trabecular meshwork proteins; NT-3, NT-4/5; hypoxanthine phosphoribosyltransferase; tyrosine hydroxylase, prostaglandin receptors, catalase and glutathione peroxidase; sequences encoding interferons, lymphokines, cytokines (cytokines acting in an anti-inflammatory manner such as TGF-β, IL-4, IL-10 or IL-13, proinflammatory cytokines such as IL-6) and antagonists thereof such as tumor necrosis factor (TNF), CD4 specific antibodies, and TNF or CD4 receptors; sequences encoding the GABA synthesizing enzyme glutamic acid decarboxylase (GAD), calcium dependent potassium channels or ATP-sensitive potassium channels; and sequences encoding thymidine kinase. Also envisioned are sequences encoding antisense nucleic acids. Other examples of polypeptides that can be encoded include dopadecarboxylase, cell adhesion molecules, interleukin-1β, superoxide dismutase, basic fibroblast growth factor, ciliary neurotroplhic factor and neurotransmitter receptors.

Nucleotide sequences encoding these polypeptides are known to those of skill in the art. For example, Abraham et al., Science 233:545, 1986, disclose the nucleotide sequence of bovine bFGF, while the-nucleotide sequence of human bFGF is disclosed by Abraham et al., EMBO J., 5:2523, 1986. Mergia et al., Biochem. Biophys. Res. Conmmun. 164:1121, 1989, provide the nucleotide sequence of the human aFGF gene. The nucleotide sequence of the rat glial cell line-derived neurotrophic factor is described by Springer et al., Exp. Neurol., 131:47, 1995. Maisonpierre et al., Genomics 10:558, 1991, provide the nucleotide sequences of human and rat brain-derived neurotrophic factor, while Arab et al., Gene 185:95, 1997, disclose the amino acid sequence of bovine brain-derived neurotrophic factor. Rat ciliary neurotrophic factor is described by Stocki et al., Nature 342:920, 1989. The nucleotide sequence of the human ciliary neurotrophic factor gene is disclosed by Negro et al., Eur. J. Biochem., 201:289 1991, Lin et al., Science, 246:1023, 1989, and by Lam et al., Gene, 102:271, 1991. Ulrich et al., Nature, 303:821, 1983, provide a comparison of human and murine coding regions of beta-nerve growth factor genes. The nucleotide sequence of bovine interleukin-1β is disclosed by Leong et al., Nucl. Acids Res., 16:9054, 1988, while Bensi et al., Gene, 52:95, 1987, provide the nucleotide sequence of the human interleukin-1β gene. All of the above-referenced are incorporated herein by reference.

DNA molecules encoding such polypeptides can be obtained by screening cDNA or genomic libraries with polynucleotide probes having nucleotide sequences based upon known genes. Standard methods are well-known to those of skill in the art. See, for example, Ausubel et al. (eds.), SHORT PROTOCOLS IN MOLECULAR BIOLOGY, 3rd Edition, pages 2-1 to 2-13 and 5-1 to 5-6 (John Wiley & Sons, Inc. 1995).

Alternatively, DNA molecules encoding growth factors can be obtained by synthesizing DNA molecules using mutually priming long oligonucleotides. See, for example, Ausubel et al., pages 8.2.8 to 8.2.13 snf pages 8-8 to 8-9. Also, see Wosnick et al., Gene, 60:115, 1987. Established techniques using the polymerase chain reaction provide the ability to synthesize DNA molecules at least two kilobases in length. Adang et al., Plant Molec. Biol., 21:1131, 1993; Bambot et al., PCR Methods and Applications 2:266, 1993; Dillon et al., “Use of the Polymerase Chain Reaction for the Rapid Construction of Synthetic Genes,” in METHODS IN MOLECULAR BIOLOGY, Vol. 15: PCR PROTOCOLS: CURRENT METHODS AND APPLICATIONS, White (ed.), pages 263-268, (Humana Press, Inc., 1993); Holowachuk et al., PCR Methods Appl., 4:299, 1995).

Preparation of Cells

Pharmaceutical Compositions

The cells of the invention can be inserted into a delivery device which facilitates introduction by e.g., injection, of the cells into the subjects. Such delivery devices include tubes; e.g., catheters, for injecting cells and fluids into the body of a recipient subject. In a preferred embodiment, the tubes additionally have a needle, e.g., a syringe, through which the cells of the invention can be introduced into the subject at a desired location. The hematopoietic progenitor cells of the invention can be inserted into such a delivery device, e.g., a syringe, in the form of a solution.

Carriers for these cells can include but are not limited to solutions of phosphate buffered saline (PBS) containing a mixture of salts in physiologic concentrations. As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi though the use of, for example, parabens, chlorobutanol, phenol, ascorbic acid, thimerosal, and the like. Solutions of the invention can be prepared by incorporating cells as described herein in a pharmaceutically acceptable carrier or diluent and, as required, other ingredients enumerated above, followed by filtered sterilization.

Cell Culture

Compositions enriched in hematopoietic stem or progenitor cells according to the invention can be maintained or expanded in culture prior to administration to a subject. Culture conditions are generally known in the art depending on the cell type. Conditions for the maintenance of CD34+ in particular have been well studied, and several suitable methods are available.

A common approach to ex vivo multi-potential hematopoietic cell expansion is to culture purified progenitor or stem cells in the presence of early-acting cytokines such as interleukin-3. It has also been shown that inclusion, in a nutritive medium for maintaining hematopoietic progenitor cells ex vivo, of a combination of thrombopoietin (TPO), stem cell factor (SCE), and flt3 ligand (Flt-3L; i.e., the ligand of the flt3 gene product) was useful for expanding primitive (i.e., relatively non-differentiated) human hematopoietic progenitor cells in vitro, and that those cells were capable of engraftment in SCID-hu mice (Luens et al., 1998, Blood 91:1206-1215). In other known methods, cells can be maintained in vitro in a nutritive medium (e.g., for minutes, hours, or 3, 6, 9, 13, or more days) comprising murine prolactin-like protein E (mPLP-E) or murine prolactin-like protein F (mPIP-F; collectively mPLP-E/IF) (U.S. Pat. No. 6,261,841). It will be appreciated that other suitable cell culture and expansion method can be used in accordance with the invention as well. Cells can also be grown in serum-free medium, described in U.S. Pat. No. 5,945,337. All of the above-referenced are incorporated herein by reference.

Cell Compositions

The invention also relates to isolated hematopoietic progenitor or stem cells as described herein, and to con positions of cells enriched in herliatopoietic progenitor or stem cells capable of migrating to the CNS of a subject, and/or capable or giving rise to microglia and/or capable of expressing a therapeutic polypeptide of interest in the CNS of a subject. Said hematopoietic progenitor or stem cells will give rise to microglia in the brain of a subject following administration by a suitable method, preferably administration outside of the CNS such as for example intravenous administration.

The invention encompasses hematopoietic progenitor or stem cell compositions specifically adapted for expressing a protein in the CNS of a mammalian subject. Said cell compositions include a huitan hematopoietic progenitor or stem cell comprising an expression vector, preferably transduced with a lentiviral vector, comprising an assembly of (1) a genetic element or elements having a regulatory role in gene expression, for example, promoters or enhancers allowing expression of a therapeutic gene operably linked thereto when the cell is present in the CNS of a subject, (2) a structural or coding sequence which is transcribed into mRNA and translated into a therapeutic polypeptide, and (3) appropriate transcription initiation and termination sequences. A therapeutic polypeptide may be a polypeptide normally expressed in the human CNS. Other examples include polypeptides capable of stimulating or encouraging the growth of CNS cells (e.g. neurons, glial cells) and polypeptides capable of inhibiting neurodegeneration. Preferably the cell composition is capable of stably expressing a therapeutic polypeptide in the CNS of a mammal. Preferably the transduced cell provides an individual with a CNS disease with a biologically active therapeutic molecule in an amount sufficient to anheliorate a symptom or feature of the CNS disease.

Administration

Delivery of the transduced cells according to the invention may be effected using various methods and includes most preferably intravenous administration by infusion as well as direct depot injection into periosteal, bone marrow and/or subcutaneous sites.

Upon administration, the cells will generally require a period of time to engraft. It is generally preferable to have the highest percentage of engraftment possible, preferred embodiments comprises achieving engraftment of at least 50%, 60%, 70%, 80%, 90%, 95%, 99%, or substantially all of the cells in the bone marrow of a subject. Achieving a high level of engraftment of hematopoietic stem or progenitor cells typically takes a period week to months.

Generally, the recipient will be treated to enhance engraftment, using a radiation or chemotheraptic treatment prior to the administration of the cells.

In general, hematopoietic progenitor or stemr cells to be administered to a subject will be autologous, e.g. derived from the subject. Nevertheless, allogeneic hematopoietic cell transplants are also envisioned, and allogeneic bone marrow transplants are carried out routinely. Allogeneic cell transplantation can be offered to those patients who lack an appropriate sibling donor by using bone marrow from antigenically matched, genetically unrelated donors (identified through a national registry), or by using hematopoietic progenitor or stem-cells obtained or derived from a genetically related sibling or parent whose transplantation antigens differ by one to three of six human leukocyte antigens from those of the patient.

Treatment

The cells and methods of this invention will be useful as providing a means for delivering a desired biologically active molecule to the CNS, e.g. to protect the endogenous affected host tissue against various neurodegenerative processes. Preferably the molecule is a secreted protein. In this aspect, a disorder relating to substantially any CNS cell population can be treated.

In another aspect, the invention can be useful in the treatment of a disorder affecting, caused by or mediated by microglia. It is contemplated that the cells can replace diseased, damaged or lost microglia in the host. Alternatively, the transplanted issue may augment the function of the endogenous affected host microglia.

As described further herein, the transplanted cells may also be genetically modified to provide a biologically active molecule that is therapeutically effective. These cells may find use in the treatment of CNS disorders, including for example metabolic disorders such as obesity having a basis in the CNS.

Thus, in one aspect, an exogenously administered active factor is provided, e.g. for providing or augmenting a function in a subject, including for the treatment of an active factor deficiency disorder and, in particular, the treatment of diseases and disorders which may be remedied by treatment with active factors, such as neurotransmitters, neuromodulators, hormones, trophic factors, cofactors, and growth factors. All these substances are characterized by the fact they are secreted by “source” cells and produce a specific change in a “target” cell or in the source cell itself. Any suitable active factor can be provided, including any of the examples provided in the section herein titled “nucleic acids”.

Deficits in active factors have been implicated in disease with very different phenotypes. For example, lack of neurotransmitter-mediated synaptic contact causes neuropathological symptoms, and can also lead to the ultimate destruction of the neurons involved.

According to the present invention, hematopoietic progenitor or stem cells which give rise to CNS cells, particularly microglia, may serve to secrete a diffusible gene product that can be taken up and used by nearby target cells. One strategy that has been pursued in animal models of neurodegenerative disease is to augment neurotransmitter function within the brain through tissue transplantation. This may prove particularly advantageous for the treatment of disorders in which diffuse delivery across the brain is required, such as in the case of Alzheimer's disease.

Two non-limiting examples of potential therapeutic uses of engineered microglia through the transplantation of genetically manipulated human CD34+ cells are further described as follows for the treatment of HIV dementia complex and Alzheimer's disease.

Manipulating Microglia in HIV Dementia Complex

Monocytes play a role in the entry of HIV into the CNS, in viral persistence in the CNS and in mediating neuronal injury. The transplantation of autologous genetically modified CD34+ cells offers the possibility to replace endogenous microglia by new microglial cells that would express mutated form of CCR5 receptors, allowing these cells to become resistant to HIV infection within the brain. Alternatively, microglia can be genetically modified to inhibit the secretion of TNFα that occurs after binding of gp120 on their CXCR4 receptors. This could be achieved by expressing mutated form of CXCR4 at the surface of microglia. Microglia can also be modified in order they express an antagonist ligand of the CXCR4 receptor or a factor that inhibit downstream signaling from CXCR4 receptor (Davis et al., J. Exp. Med., 186:1793-1793, 1997 incorporated herein by reference).

Manipulating Microglia in Alzheimer's Disease

The presence of microglia in senile plaques offers a number of targets for therapeutic intervention. Most of them could be achieved through the replacement of endogenous microglia by new microglia after autotransplantation of genetically modified CD34+ cells. These targets include: 1) the signaling steps that lead to neuronal damage because microglia are activated in the presence of Aβ-containing plaques; 2) the up-regulation of Aβ clearance by nucroglia; 3) the interruption of Aβ binding to microglia; 4) the production of survival neuronal factors.

In one aspect the invention involves a method of treatment comprising the interruption of a signaling inflammatory cascade that leads to neuronal damage by providing a hematopoietic stem or progenitor cell capable of giving rise to microglia. Microglia can be provided whose expression of C1 inhibitor is up-regulated allowing the inactivation of complement pathway, or microglia can be provided that express an inhibitor of COX-2 activity. A number of retrospective clinical observations, as well as epidemiological data, have suggested that anti-inflammatory drugs may offer protection against AD.

In one aspect the invention involves a method of treatment comprising up-regulation of Aβ processing by providing a hematopoietic stem or progenitor cell capable of giving rise to microglia. Microglia can be made to overexpress the cytokine TGF-β1 (Wyss-Coray T. et al., Nat. Medecine, 7:612-618, 2001). TGF-β1 may directly stimulate microglia to phagocytose Aβ peptides or alternatively induce the secretion of Aβ-binding proteins by astrocytes, which facilitate microglial phagocytosis.

In another aspect, the invention involves a method of treatment comprising preventing Aβ binding to microglia by providing a hematopoietic stem or progenitor cell capable of giving rise to microglia. Aβ binding to microglia may activate microglia and hence leads to neuronal damage. This process can be inhibited by providing microglia that would secrete HHQK-like peptides that, in turn, will impede the binding of Aβ peptides to microglia type-A macrophage scavenger receptor. This strategy offers the advantage to suppress only the toxicity that occurs during Aβ-dependent activation of microglia without impairing their other immune functions.

In other strategies, microglia can be genetically modified to express neuronal trophic factors. NGF is promising given it protects cholinergic neurons from axotomy-induced cell death in fimbria-fornix lesion models, reverses age-associated atrophy of cholinergic cell bodies and improves spatial navigation, memory and learning in mice.

Manipulation Neuronal NF-κB Activation in Microglia to Increase Neuroprotection

Based on work in animal models, manipulation of NF-κB signaling may be valuable in treating several neurodegenerative disorders, including Alzheimer's disease (AD) and Parkinson's disease (PD) (Mattson M. P. and Camandola S. J., Clin. Invest., 107:247-254, 2001).

Functional NF-κB complexes (p50, P65 and IκBα) are present in microglia and neurons. NF-κB influences the expression of a complex array of genes in the CNS, and in general, theses genes serve important functions in cellular responses to injury. NF-κB is activated by signals that activate IκB kinase (IKK), resulting in phosphorylation of IκBα. This targets IκBα for degradation in the proteosome and frees p65-p50 dimer, which then translocates to the nucleus and binds to consensus κB sequences in the enhancer region of κB-responsive genes. In general, it appears that genes activated by NF-κB in neurons protect them against degeneration whereas activation of NF-κB in macroglia promotes neuronal degeneration.

In AD, TNFα can protect neurons against Aβ-induced death via a NF-κB mediated mechanism. α-secretase-derived form of secreted amyloid precursor protein (sAPPα is potently excitoprotective and antiapopotic in CNS neurons. NF-κB activation following exposure to sAPPα is correlated with increased resistance of neurons to metabolic and excitotoxic insults. As a result of aberrant proteolytic processing of βAPP, levels of sAPPα may be decreased. It seems likely that activation of NF-κB in neurons associated with amyloid deposit is a cytoprotective response. On the other hand, the increased levels of membrane lipid peroxidation that occur in neurons degenerating in AD may endanger neurons by suppressing NF-κB activation. This is the case of 4-hydroxynonenal which inhibits NF-κB activation. Moreover, prostate apoptosis response-4 (Par-4), a proapoptotic protein implicated in the pathogenesis of neuronal degeneration in AD, strongly suppresses NF-κB activation in cultured neural cells.

Immunohistochemical analyses of brain sections from PD patients show a 70 fold increase of nuclear p65 NF-κB protein in dopaminergic neurons of substantia nigria. Spinal cords of patients with amyotrophic lateral sclerosis show increased NF-κB activation in astrocytes associated with degenerating motor neurons. In both diseases, the increased NF-κB activity in the affected neurons may represent an early protective response to ongoing oxidative stress and mitochondrial dysfunction.

Exitotoxic and ischemic injury to neurons is mediated in part by dysregulation of cellular calcium homeostasis resulting in a prolonged elevation of intracellular calcium levels. Activation of NF-κB in neurons can stabilize intracellular calcium levels under ischemia-like conditions. This may result from induction of several different genes, including those encoding calcium-binding proteins (like calbindin) and glutamate receptor subunits.

Although activation of NF-κB in neurons can prevent apoptosis in these cells, NF-κB activation in microglia may indirectly lead to apoptosis of other cells by promoting production of cytotoxic agents such as nitric oxide. Cytokine-mediated activation of microglia may explain the ability of inhibitors of NF-κB to protect against cell damage in certain experimental paradigms that involve an inflammatory responses in which microglia is activated. Microglial activation is associated with a marked increase in COX-2, an oxyradical-generating enzyme, and agents that inhibit NF-κB activation can suppress LPS (liposaccharide)-induced COX-2 expression.

The transplantation of autologous genetically modified CD34+ cells offers the possibility to replace endogenous microglia by new microglial cells that could activate NF-κB in neurons. On the other hand, it is also possible to replace endogenous microglia by genetically modified microglia in which activation of NF-κB pathway leading to deleterious effects is inhibited.

In one approach, the invention encompasses activating NF-κB in neurons by transplanting genetically modified CD34+ cells whose derived-microglia will secrete sAPPα or activity-dependent neurotrophic factor (ADNF), either of which are good candidates to activate NF-κB in neurons.

In another strategy, genetically modified CD34+ cells that will differentiate into microglia secreting at a low and regulated level heat-shock proteins can be transplanted. Neurons exposed to low level of heat-shock proteins can be preconditioned through NF-κB activation. Neuronal preconditioning increases resistance of neurons to various oxidative, metabolic and excitotoxic insults in experimental models relevants to AD, PD and Huntington's disease.

In a further strategy for inactivating NF-κB pathway in microglia, genetically modified CD34+ cells can be transplanted, the cells giving rise to microglia expressing proteosome inhibitors (which inhibit NF-κB activation by preventing degradation of IκBα), peptides or oligonucleotide inhibitors that block DNA-binding activity of p50/p65 dimers on consensus κB sequences.

Manipulating Microglia to Express Neurotophic Factors in AD, PD and Multiple Slcerosis

Neurotrophic factors are secreted peptides that are of potential values in several neurodegenerative diseases, including AD and PD (Siegel and Chauhan, 2000). These diffusible proteins act via retrograde signaling promoting neuronal surviving. For most of them, their systemic injection lead to serious side effects that limit their clinical use. One possibility to circumvent these limitations would be to transplant genetically CD34+ cells whose derived-microglia will secrete neurotrophic factors, likely in combination since many studies have demonstrated that combined administration of neurotropic factors is often synergistic. In multiple sclerosis, remyelinating “shadow” plaques can be observed in the early acute phase of the disease but the rate of remyelination is limited. One could envisage to transplant genetically CD34+ cells whose derived-microglia will secrete growth factors that promote differentiation of oligodendrocytes precursors and survival of oligodendrocytes (reviewed in Diemel et al., 1998).

### EXAMPLES

### Example 1

### Transplantation of Human Modified CD34+ Cells can Differentiate into Brain Microglia Expressing a Transgen. Materials and Methods

Lentiviral Vector

TRIP-ΔU3-EF1α-ALD lentiviral vector was constructed by replacing the enhanced green fluorescent protein (EGFP) cassette (BamHI/KpnI) from the previously described TRIP-ΔU3-EF1α-EGFP lentiviral vector (Sirven, A. et al., Blood 96, 4103-4110, 2000, and Sirven, A. et al., Mol. Ther., 3, 438-448, 2001) by a BamHI-EcoRI fragment containing the coding sequence of the human ALD cDNA (Mosser, J. et al., Nature, 361, 726-730, 1993). This self-inactivating (SIN) vector where the U3 region of the 3′LTR is deleted to improve the safety of the vector system includes the central polypurine tract (cPPT) and the central termination sequence (CTS) (Zennou, V. et al., Cell, 101, 173-185, 2000) that increases the gene transduction efficiency in human CD34+ hematopoietic cells (Sirven et al., 2000). The expression of the ALD gene is driven by the elongation factor 1α (EF 1α) promoter that allows homogeneous expression in all hematopoietic cell types and particularly in NOD-SCID repopulating cells (Sirven et al., 2001).

Preparation of High-Titer Virus Vector

Lentivirus vectors were generated by transient calcium phosphate co-transfection of 293T cells by the vector plasmid, an encapsidation plasmid lacking all accessory HIV-1 proteins (p8.91) and a VSV (vesicular stomatitis virus) envelope expression-plasmid (pHCMV-G), as previously described (Zennou et al., 2000).

Vector particles were normalized according to both p24 (HIV-A capsid protein) content of supernatants (Zennou et al., 2000) and measurement of infectious titer on murine 3T3 cells (Cartier, N. et al., Proc. Natl. Acad. Sci. USA, 92, 1674-1678, 1995). Viral titers varied from 5.10⁸ to 10⁹ IU/ml.

Isolation of ALD CD34⁺ Cells

CD34+ cells were isolated from granulocyte colony-stimulating factor (G-CSF)-mobilized peripheral blood from ALD patients according to approved institutional guidelines. CD34⁺ cells were purified by immuno-magnetic selection (Miltenyi Biotec, Paris, France) as previously described (Sirven, 2000 and 2001). Fluorescent activating cell sorting (FACS) analysis performed on a FACStar (Becton Dickinson) showed over 90% purity of the CD34+ population. CD34⁺ cells were then stored in liquid nitrogen before use.

Transduction Protocol

CD34⁺ cells were plated at 10⁶ cells/ml in serum free medium (Stem Cell Technologies, Vanvouver, Canada) in the presence of 4 recombinant human cytokines: 10 ng/ml stem cell factor (SCF) (Amgen, Neuilly-sur-Seine, France); 10 ng/ml Flt3-Ligand (FL) (Immunex, Seattle, USA); 10 ng/ml interleukin (IL)-3 (Novartis France, Rueil-Malmaison, France) and 10 ng/ml pegylated-megacaryocyte-growth and differentiation factor (PEG-MGDF hereafter named TPO) (Kirin Brewery, Tokyo, Japan). Lentiviral vector particles were added twice at 0 and 12 hour at multiplicity of infection (MOI) of 5. At 36 hours, transduced and non-transduced CD34+ cells were washed and cultured for 72 hours in H5100 long term culture medium (StemCell Technology, Vancouver, Canada) on MS5 stromal cells. Expression of the human ALD protein (ALDP) was analyzed by immunocytofluorescence (Cartier et al., 1995; Fouquet, F. et al., Neurobiol. Dis., 3, 271-285, 1997; and Doerflinger N. et al., Hum. Gene Ther., 9, 1025-1036, 1998).

Hematopoietic Cell Cultures

Colony forming cells (CFCs) and long-term culture-initiating cells (LTC-ICs) were assayed as described (Sirven et al., 2000 and 2001).

Bulk and 1/10/50 per well long-term culture (LTC) cells were studied separately. After 5 weeks, LTC cells were plated on methycellulose plates and colonies were assessed 15 days later for ALDP expression.

Lymphoid (B, NK) and myeloid (granulo-monocytic) differentiation was assessed on MS5 stronial cells in the presence of SCF, FL, TPO, IL-15 and IL-2 as described (Sirven et al., 2000 and 2001).

Cells were phenotyped by FACS after 3-4 weeks of culture, using the following mouse monoclonal antibodies (mAbs): CD19-PE (phycoerythrin) (Becton Dickinson) for B lymphocytes; CD15-PE and CD14-PE (PharMingen, Pont de Claix, France) for granulocytes and macrophages; CD56-PE-Cy5 (Immunotech, Villepinte-Roissy CDG, France) for NK cells and CD34-PE-Cy5 (Immunotech, Villepinte-Roissy CDG, France). Non-specific staining was detected using irrelevant mouse IgG1 and IgM mAbs.

Expression of ALDP was scored using immunocytochemistry with an anti-human ALDP antibody (Fouquet et al., 1997 and Doerflinger et al., 1998) in CFCs, LTC cells and cells from LTC giving rise to CFCs (LTC-Ics).

Expression of ALDP in monocytes-macrophages derived from lympho-myeloid cultures and LTC cells was analysed with polyclonal anti-human ALDP (Fouquet et al., 1997 and Doerflinger et al., 1998) and monoclonal anti-CD68 KP1 (Dako, Carpinteria, CA) antibodies after incubation with horse anti-mouse IgG (H+L) antibody directly conjugated to fluorescein (Vector Laboratories) and biotinylated anti-rabbit IgG antibody and further incubation with Cy3-conjugated streptavidine (Chemicon).

Transplantation of Transduced ALD CD34⁺ Cells into NOD/SCID Mice

Immediately after transduction, 1.5-10⁶ ALD CD34⁺ cells were intravenously injected into sub-lethally irradiated NOD-LtSz-scid/scid NOD/SCID) mice (3 Gy, at 0.43 Gy/mn; in a X-ray Phillips RT250 irradiator). Eighteen weeks later, bone marrow cells were harvested from recipient mice and the presence of human cells was assessed in individual mice by FACS using mouse anti-human CD45 (Inmmunotech, Villepinte-Roissy CDG, France), CD38, CD19, CD14-PE and CD34-PE-Cy5 mAbs.

Human ALDP expression was studied by immunocytochemistry on at least 500 bone marrow cells with an antibody that does not cross react with the mouse ALDP (Fouquet et al., 1997).

Human CD34⁺ cells were purified from the bone marrow of two transplanted NOD/SCID mice and cultured in lympho-myeloid conditions (Sirven et al., 2000 and 2001).

Brain Immunohistochemistry

Deeply anesthetized animals were sacrificed. Brain was removed, frozen into isopentane and stored at −80° C. until analysis. Serial sections (10 μm thick) were cut at −17° C. using a cryostat, fixed in 4% formaldehyde for 15 min and permreabilized in PBS-Triton X-100, 0.1%. Immunostaining of ALDP expressing cells and microglia was performed with anti-human ALDP antibody and Ricinus Conrnunis Agglutin (RCA) as described (Fouquet et al., 1997). Cell nuclei were stained with 4′,6-diamidino-2-phenylindole (DAPI). Appropriate filters for each or combined fluorochrome were used on a light microscope equipped for fluorescence (Nikkon E600).

In Situ Hybridization Histochemistry

Serial brain sections (10 μm thick) were cut at −17° C. using a cryostat, fixed in 4% formaldehyde for 15 min and permeabilized in PBS-Triton X-100, 0.1%.

Non radioactive in situ hybridization was performed using a specific human Alu oligodeoxynucleotide probe labeled in 5′ with digoxigenin (Wilkinson, D.G. (ed). In Situ Hybridization. A practical Approach. Oxford University Press, New York, 1992).

After denaturation at 75° C. for 20 minutes, brain slides were prehybridized in wet steamnroom chambers at 45° C. for 1.5 h. Slides were then placed overnight at 45° C. in the hybridization solution containing the probe (0.02 pmol/μl).

After 5 washes, antibody against digoxigenin conjugated to alkaline phosphatase (1:2000 dilution, Roche Diagnostics) was added and digoxigenin was revealed with nitroblue tetrazolium/5-bromo-4-chloro-3-indolyl phosphate (NBT/BCIP) (Promega). Labeled cells were counted on six sagittal brain sections of each transplanted mice.

### Example 2

### Transplantation of Human Modified CD34+ Cells can Differentiate into Brain Microglia Expressing a Transgene. Results

Lentiviral Vector-Mediated ALD Gene Transfer into ALD Deficient CD34+ Cells

A 36-hour long transduction protocol characterized by the absence of cytokine prestimulation and low-cytokine serum-free medium was used. This allows effective transduction of CD34+ by lentiviral vectors in the late G1 phase (Sutton, R. E. et al., J. Virol., 73, 3649-3660) but avoid terminal differentiation.

CD34+ cells from 3 ALD patients whose ALD gene mutation leaded to a complete absence of ALD protein were used. After wash-out, cells were incubated for 72 hours in long-term culture medium without cytokines. Transduction efficacy was then analysed by the expression of ALD protein using immunocytochemistry. 37.5 to 56.5% (mean 47.2%) of ALD cells expressed ALDP (Table 1).

To examine the transduction efficiency in colony-forming cells (CFCs), ALD deficient CD34+ cells were immediately plated on methylcellulose after transduction and cultured for 14-16 days. The number of individual CFC that expressed ALDP ranged from 32.5 to 39% (Table 1) (mean 36.6%).

No difference either in plating efficiency or CFU-GM/BFU ratio was observed in transduced and non-transduced cells (data not shown).

In vitro Analysis of Transduced ALD Deficient Hematopoietic Cells

In a perspective of CNS gene therapy, the main goal of gene transfer in human hematopoieitic stem cells is to target immature stem cells with proliferating and differentiating potentials in monocytes/macrophages in peripheral tissues and microglia in brain. To demonstrate that the TRIP-ΔU3-EF1α-ALD lentiviral vector was able to transduce ALD gene into such cells, different approaches were used.

First, transduced ALD deficient CD34+ cells were cultured in conditions that promote lympho-myeloid differentiation (Sirven et al., 2000 and 2001). B, NK and myeloid cells were obtained. Monocytes-macrophages were identified with an anti-CD68 antibody and double immunostaining with anti-ALDP antibody. ALD CD34+ cells transduced with 35% efficacy (ALD patient #2) could differentiate in CD68+ cells and 15% of these cells expressed ALDP.

Second, transduced CD34+ cells from 2 ALD patients (#1 and 2) were maintained in long-term culture (LTC) for 5 weeks. ALDP was expressed in 30% of 3000 cells that were studied at the end of the 2 LTC with no variation of this percentage between LTC experiments. Given that erythroid precursors comprise 35% of all bone marrow cells and do not express ALDP, this allows to estimate that 46% of all LTC cells expressed ALDP after 5 weeks of culture.

LTC cells were then plated on methylcellulose and CFU-GM colonies were individually and randomly picked and scored for ALD expression. From a total of 90 CFU-GM colonies (from the 3 LTC of patients #1 and #2), 44% expressed ALDP (Table 1), indicating that 44% of these transduced cells were early hematopoietic progenitors.

To determine the transduction efficacy in LTC-IC cells (LTC-Ics), LTC of transduced ALD CD34+ cells was performed in 96-well plates by plating one, ten or fifty CD34+ cells per well. After 5 weeks, cells from each well were plated on methylcellulose and the number of CFU-GM colonies was scored after 15 days. The percentage of wells giving rise to CFU-GM colonies is representative of the LTC-IC frequency of planted cells. 20% of transduced ALD deficient CD34+ cells were LTC-ICs, in agreement with that observed with non transduced peripheral blood CD34+ cells.

ALDP expression was scored in CFU-GM colonies derived from ten-cell-wells. Colonies from each methylcellulose plate were pooled and analysed. ALDP-expressing cells were found in every plate, meaning that at least 50% of LTC-ICs have been transduced (20% of 10 transduced ALD CD34+ cells were LTC-IC, i.e. colonies obtained in each plate originated from 2 cells. In each analysed plate, we found colonies expressing ALDP, meaning that at least one of the two LTC-IC from which they derived expressed ALDP).

TABLE 1
Expression of ALD protein (ALDP) in human ALD deficient
CD34+ cells after lentiviral-mediated ALD gene transfer.

| | % of cells | | |
|---|---|---|---|
| | expressing | % of individual | % of individual |
| ALD | ALDP 72 hours | CFU-GM colonies | CFU-GM colonies |
| Patient | after transduction | expressing ALDP | derived from LTC |
| #1 | 56.5 ± 13.1 | 38.5 ± 16.1 | 39; 43 |
| | (n = 4) | (n = 3) | (n = 2) |
| #2 | 37.5 ± 3.5 | 39 ± 8.5 | 50 |
| | (n = 2) | (n = 2) | (n = 1) |
| #3 | 47.5 ± 3.5 | 32.5 ± 3.5 | ND |
| | (n = 2) | (n = 2) | |

*results are expressed as mean ± SD of n performed experiments.
ND: not done.

Functional Correction of ALD Biochemical Defect in Hematopoietic Cells in vitro

ALD is biochemically characterized by the accumulation of VLCFAs that involves mainly hexacosanoic (C_{26:0}) acid whereas the concentration of docosanoic (C_{22:0}) acid remains normal. The C_{26:0}/C_{22:0} ratio thus reflects the ability of cells to metabolize VLCFA in the presence of functional ALD protein (Dubois-Dalcq, M. et al., Trends Neurosci., 22, 4-12, 1999).

Table 2 shows that the C_{26:0}/C_{22:0} ratio decreased proportionally to the percentage of ALDP expression in ALD CD34+ cells, 72 hours after transduction, in CFU-GM derived cells and in transduced ALD CD34+ cultured for 5 weeks (LTC). The correction of C_{26:0}/C_{22:0} ratio was greater than expected with respect to the number of cells expressing ALDP suggesting that overexpression of ALDP leads to increase VLCFA degradation (Doerflinger et al., 1998). These results indicate that lentiviral-vector encoded ALD protein was functional in peroxisomes of transduced hematopoietic ALD cells.

TABLE 2
Correction of very-long chain fatty acid (VLCFA) metabolism in ALD
deficient CD34+ cells after transduction with a lentiviral
vector and in derived CFU-GM colonies and LTC cells.

| | | | | % of transduced | % of |
|---|---|---|---|---|---|
| | Observed C_{26:0}/C_{22:0} | | | ALD cells | biochemically |
| | | ALD cells | | expressing | corrected |
| | Control cells | Non-transduced | Transduced | ALDP | ALD cells |
| CD34+ cells | 0.041 ± 0.018 | 0.0192 ± 0.0421 | 0.118 ± 0.016 | 40 | 48 |
| | (n = 4) | (n = 2) | (n = 2) | | |
| CFU-GM | 0.042 ± 0.016 | 0.121 | 0.072 | 45 | 63 |
| derived cells | (n = 2) | (n = 1) | (n = 1) | | |
| Cells derived | 0.021 ± 0.012 | 0.113 | 0.088 | 16.5 | 26 |
| from 5-week | (n = 2) | (n = 1) | (n = 1) | | |
| LTC | | | | | |

Engraftment of Transduced ALD Deficient CD34+ Cells in NOD/SCID Mice

Because long-term in vivo transplantatibility of human hematopoietic cells in NOD/SCID mice is considered a hallmark of cell immaturity (Dao, M. A. et al., Cur. Opin. Mol. Ther., 1, 553-557, 1999), we injected 10⁶ to 1.5.10⁶ ALD CD34+ cells immediately after transduction into 5 NOD/SCID mice.

Eighteen weeks after transplantation, mice were sacrificed and human hematopoietic engraftment was analyzed by FACS of bone marrow cells with anti-human CD45 antibody. Two out of five NOD-SCID mice were engrafted with transduced ALD deficient CD34+ cells in proportion ranging from 25% to 75% (Table 3). Human ALDP was expressed in 30 and 85% of bone marrow cells from recipient mice #3 and 8 respectively (Table 3).

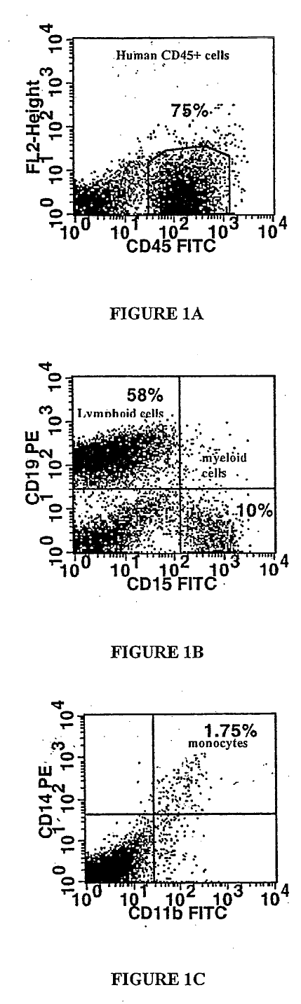

The bone marrow cells of NOD/SCID mouse #3 engrafted with 75% CD45+ human cells (Table 3; FIG. 1A) were phenotyped with specific human antibodies against CD11b, CD14, CD15 and CD19 antibodies. 58% of human CD45+ cells were B lymphocytes (CD19+) (FIG. 1B), 10% myeloid cells (CD15+) (FIG. 1B) and 1.75% monocytes (CD14+, CD 11+) (FIG. 1C).

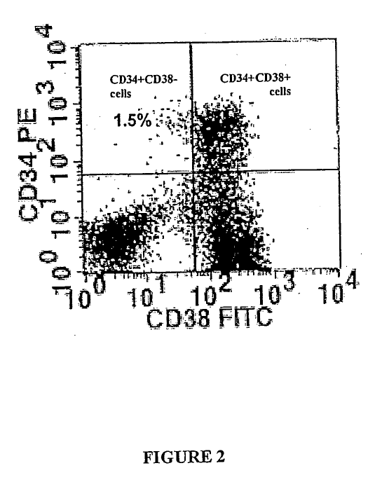

Bone marrow from mouse #3 contained human CD34+/CD38− cells (FIG. 2), indicating that early human hematopoietic progenitor cells were maintained in vivo.

Bone marrow CD34+ cells from mouse #3 were sorted by flow cytometry and cultured in conditions that promote lympho-myeloid differentiation (Sirven et al., 2000 and 2001). CD68 positive cells present in this culture expressed ALDP indicating that long-term NOD/SCID repopulating cells derived from transduced ALD deficient CD34+ cells were able to differentiate into monocytes/macrophages and express recombinant ALDP in bone marrow.

Transduced Human Deficient ALD CD34+ Cells can Differentiate into Microglia and Express ALDP in the Brain of NOD/SCID Mice

In situ hybridization of brain slices from mouse #3 and #8 showed the presence of human Alu positive cells in brain and cerebellum (FIGS. 3A and 3B). 70±12 Alu positive cells per slice were present in the brain from mouse #3 and 5±2 in the brain slices from mouse #8 (Table 3).

Double immunostaining with antibodies against RCA (in green) and human ALDP (Cy3 in red) revealed the presence of human microglial cells that expressed ALDP in the brain both recipient NOD-SCID mice (FIG. 3C). 15±4 ALDP positive cells per slice were present in the brain from mouse #3 and 5±0.5 in the brain slices from mouse #8 (Table 3). These numbers are close to what would be expected when taking into account the percentage of engraftment and human CD45+ ALDP positive cells in the bone marrow from these 2 NOD-SCID mice. This demonstrates that ALDP was expressed up to 4 months in human brain microglia present in the brain of NOD/SCID mice that originate from transduced ALD deficient CD34+ cells.

Altogether, these data demonstrate that, in a model of xeno-transplantation (the NOD-SCID mouse), human CD34+ can be genetically modified ex vivo to express a “therapeutic” protein and that these cells can differentiate in vivo into microglia and express in long term (4 months) a genetically engineered “therapeutic” protein after bone transplantation.

TABLE 3
Analysis of ALDP positive cells in bone marrow
and brain from NOD-SCID mice

| | | % of | | | |
|---|---|---|---|---|---|
| | | human | | Expected | Expected |
| | % of | CD45+ | Number | number | number |
| | human | cells | of Alu | of ALDP | of ALDP |
| | CD45+ | expressing | positive | positive | positive |
| | cells | ALDP | cells | cells | cells |
| NOD-SCID | in bone | in bone | in brain | in brain | in brain |
| Mouse | marrow | marrow | per slice | per slice | per slice |
| #3 | 75 | 30 | 70 ± 12 | 21 | 15 ± 4 |
| #8 | 25 | 80 | 5 ± 2 | 4 | 4 |

Throughout this application, various publications are, referenced. The disclosures of these publications in their entireties are hereby incorporated by reference into this application in order to more fully describe the state of the art to which this invention pertains.

1-20. (canceled)

21. A composition for the treatment of a subject affected by or susceptible to being affected by a CNS disorder, wherein the composition comprises a population of human cells enriched in human cells that can express human CD34, wherein at least of portion of the cells comprises a nucleic acid of interest, and wherein the composition comprises the human cells in an amount sufficient to migrate to the CNS of a human subject and express the nucleic acid of interest in the CNS of the human subject when intravenously administered to the subject.

22. The composition according to claim 21, wherein cells in the composition are capable of giving rise to microglia in the CNS when administered to the human subject.

23. The composition according to claim 21, wherein the nucleic acid of interest encodes a polypeptide of interest, and wherein the composition comprises hematopoietic progenitor cells or hematopoietic stem cells isolated from cells obtained from the human subject, and wherein the nucleic acid encoding the polypeptide of interest has been introduced into the isolated hematopoietic progenitor cells or hematopoietic stem cells under conditions that result in the expression of the polypeptide of interest at a level that provides a therapeutic effect in the human subject.

24. The composition according to claim 21, wherein the composition contains the human cells in an amount sufficient to reduce the severity of central nervous system damage or symptoms of a central nervous system disorder in the subject.

25. The composition according to claim 23, wherein at least of portion of the cells are recombinant cells comprising the nucleotide sequence encoding the polypeptide operably linked to expression control elements.

26. The composition according to claim 25, wherein the composition contains the human cells in an amount sufficient to reduce the severity of central nervous system damage or symptoms of a central nervous system disorder in the subject.

27. The composition according to claim 21, wherein the administered cells are hematopoietic progenitor or hematopoietic stem cells that can differentiate into microglia cells.

28. The composition according to claim 21, wherein at least 20% of cells in the composition express the CD34+ marker.

29. The composition according to claim 21, wherein the cells are isolated from the human subject.

30. The composition according to claim 22, wherein the cells are recombinant cells comprising a nucleic acid of interest.

31. The composition according to claim 29, wherein at least a portion of the cells are cells that can express CD34 and are transduced with a vector comprising the nucleic acid of interest operably linked to a promotor capable of effecting the expression of the nucleic acid of interest in the cells.

32. The composition according to claim 31, wherein at least a portion of the cells are transduced with a viral vector.

33. The composition according to claim 32, wherein the viral vector is a lentiviral vector.

34. The composition according to claim 23, wherein the hematopoietic progenitor or hematopoietic stem cells express the CD34+ marker or are capable of differentiating into cells expressing the CD34+ marker.

35. The composition according to claim 24, wherein the cells that can express human CD34 are hematopoietic progenitor cells or hematopoietic stem cells.

36. The composition according to claim 21, wherein the nucleic acid of interest encodes a non-secreted or a secreted protein.

37. A method of treating a subject affected by or susceptible to being affected by a CNS disorder, wherein the method comprises administering to the subject a composition comprising a population of human cells enriched in human cells that can express human CD34, wherein at least of portion of the cells comprises a nucleic acid of interest, and wherein the composition is administered to the subject in an amount sufficient to migrate to the CNS of a human subject and express the nucleic acid of interest in the CNS of the human subject when intravenously administered to the subject.

38. The method as claimed in claim 37, wherein the subject to be treated is pretreated in order to enhance engraftment of the composition comprising the cells.

39. The method as claimed in claim 37, wherein the CNS disorder, which affects or which is susceptible to affect the subject, is characterized by diffuse neurodegeneration.

40. The method as claimed in claim 37, wherein the CNS disorder is Alzheimer's disease.

41. The method as claimed in claim 37, wherein the administered cells are autologous to the subject to be treated.

42. The method as claimed in claim 37, wherein cells in the composition are capable of giving rise to microglia in the CNS when administered to the human subject.

43. The method as claimed in claim 37, wherein the nucleic acid of interest encodes a polypeptide of interest, and wherein the composition comprises hematopoietic progenitor cells or hematopoietic stem cells isolated from cells obtained from the human subject, and wherein the nucleic acid encoding the polypeptide of interest has been introduced into the isolated hematopoietic progenitor cells or hematopoietic stem cells under conditions that result in the expression of the polypeptide of interest at a level that provides a therapeutic effect in the human subject.

44. The method as claimed in claim 27, wherein the composition contains the human cells in an amount sufficient to reduce the severity of central nervous system damage or symptoms of a central nervous system disorder in the subject.

45. The method as claimed in claim 27, wherein at least a portion of the cells are recombinant cells comprising the nucleotide sequence encoding the polypeptide operably linked to expression control elements.

46. The method as claimed in claim 27, wherein the administered cells are hematopoietic progenitor or hematopoietic stem cells that can differentiate into microglia cells.

47. The method as claimed in claim 27, wherein at least 20% of cells in the composition express the CD34+ marker.

48. The method as claimed in claim 27, wherein the cells are isolated from the human subject.

49. The method as claimed in claim 27, wherein at least a portion of the cells are transduced with a viral vector.

50. The method as claimed in claim 49, wherein the viral vector is a lentiviral vector.

51. The method as claimed in claim 27, wherein the nucleic acid encodes a non-secreted or a secreted protein.
